(19) United States
(12) Patent Application Publication
(10) Pub. No.: US 2025/0345609 A1
(43) Pub. Date: Nov. 13, 2025

(54) SYSTEM, SOFTWARE AND METHODS OF USING SOFTWARE FOR TREATING AND MODELING HEART DISEASE

(71) Applicant: Lehigh University, Bethlehem, PA (US)

(72) Inventors: Yuyu YAO, Hillsboro, OR (US); Mayuresh V. KOTHARE, Slatington, PA (US)

(73) Assignee: Lehigh University, Bethlehem, PA (US)

(21) Appl. No.: 18/880,107

(22) PCT Filed: Jun. 30, 2023
(86) PCT No.: PCT/US2023/026790

§ 371 Date: Dec. 30, 2024

Related U.S. Application Data
(60) Provisional application No. 63/357,238, filed on Jun. 30, 2022.
(60) Provisional application No. 63/500,191, filed on May 4, 2023.

(51) Int. Cl. A61N 1/36

(52) U.S. Cl. CPC A61N 1/36139; A61N 1/36117; A61N 1/3615; A61N 1/36171

(57) ABSTRACT

The disclosure relates to a system comprising a device that includes electrodes and a sensor operably connected in electrical communication with a controller housing a computer program product that calculates heart rate and mean arterial blood pressure in the circulatory system of a subject by the sensor being placed at or proximate to the cardiac tissue of the subject. Electrodes on the device stimulate the vagal nerve to restore heart rate and arterial blood pressure to normal levels if an abnormality is detected.

### CROSS-REFERENCE TO RELATED APPLICATIONS

This application claims priority to U.S. Provisional Application Nos. 63/357,238, filed Jun. 30, 2022, and 63/500,191, filed May 4, 2023, each of which are incorporated by reference in their entirety.

### GOVERNMENT SUPPORT CLAUSE

This invention was made with government support under OT20D030535 awarded by the National Institutes of Health. The government has certain rights in the invention.

### FIELD OF INVENTION

The disclosure relates to a system comprising computer program product or software that monitors mean arterial pressure and heart rate in a subject and that applies a pulse of current to an electrode operably linked to a controller comprising the computer program product when measurements correlated to mean arterial pressure and heart rate of the subject correspond to an abnormal value. Embodiments of the disclosure include methods comprising analyzing value input from measurements of the subject's circulatory system and administering electrical pulses to the subject to treat abnormal heart rate and abnormal mean arterial pressure.

### BACKGROUND

Several effective approaches to modeling cardiovascular vagal response have been reported for implementing closed loop control of VNS to determine optimal stimulation parameters in animal studies. For example, standard proportional-integral controllers were designed to regulate heart rate of dogs [8], [9], pigs [10], and rats [11]. Another study used a model-based framework to tune the parameters of a proportional-integral controller before applying it on sheep to control heart rate [12]. The previously discussed controllers were designed as single-input-single-output systems. A more recent controller based on state-transition models was developed to manipulate multiple VNS parameters [13]. However, the accuracy of this controller is dependent on the number of states, which is limited by the memory of the implantable device. Our group previously developed a nonlinear model predictive control (NMPC) algorithm, which manipulates pulse frequency and pulse amplitude at multiple stimulation locations to control the heart rate and blood pressure simultaneously.

Significant challenges prevent the NMPC and the other above-identified systems from becoming commercialized. One of the challenges associated with this application of NMPC includes the development and validation of a predictive cardiac model to be used in NMPC. The variety of cardiac models in the literature, ranging from an individual cardiac myocyte to the whole circulatory system, make it difficult to decide which equations are accurate enough in capturing the cardiac dynamical response. It is also difficult to integrate parameters to these nonlinear equations. Another challenge involves the high computational cost. The numerical complexity of NMPC and the other above-systems prevent a timely, global solution to the resulting nonlinear optimization problem within real-time requirements.

### SUMMARY OF EMBODIMENTS

The disclosure relates to a device and a method of using the system for predicting a control level of heart rate (HR) and mean arterial blood pressure (MAP). The disclosure relates to a device and a method of using the system for predicting a real-time level of heart rate (HR) and mean arterial blood pressure (MAP). In some embodiments, the disclosure relates to a system comprising a device that comprises a sensor capable of detecting HR and MAP of a subject. In some embodiments, the device comprises a computer program product encoded on a computer-readable storage medium with instructions for measuring MAP and HR in a cardiac cycle of a subject, predicting a control cardiac response of a circulatory loop, measuring the real-time levels of the MAP and HR of a subject, and then calculating a desired adjustment value for the stimulus parameters of frequency, amplitude, and location, corresponding to a difference between an estimated optimal or healthy values of MAP or HR and the measured values of MAP or HR in the subject. In some embodiments, the device comprises a computer program product encoded on a computer-readable storage medium with instructions for measuring MAP and HR in a cardiac cycle of a subject, predicting a control cardiac response of a circulatory loop, measuring the real-time levels of the MAP and HR of a subject, and then calculating a desired adjustment value corresponding to MAP and HR, which are two values that are the difference between an estimated optimal or healthy values of MAP or HR and the measured values of MAP or HR in the subject.

In some embodiments, the computer program product comprises instructions that further command an electrode to provide an electrical pulse to the vagal nerve of the subject with a magnitude equivalent to the desired adjustment value. In some embodiments, the device comprises a first, second and third electrode that can be placed in three distinct locations along or proximate to the vagal nerve of the subject. When embedded or transplanted into the subject and upon receiving a command from the computer program product, the first, second and third electrodes stimulate the vagal nerve of the subject with an amplitude and frequency of an electrical pulse that, in sum, are the desired adjustment values corresponding to each of the HR and the MAP, respectively. The objective of the device is to correct for abnormal HR and/or MAP in a subject in need of treatment. In some embodiments, the disclosure relates to a system comprising an implantable device comprising: (i) a sensor capable of detecting HR and MAP of a subject; (ii) at least one electrode in electrical communication with an electricity source; and (iii) a battery source. In some embodiments, the system further comprises a controller and a computer storage memory in operable connection with the device. In some embodiments, the controller is positioned within the device and operably connected in electrical communication to the electrode, the electricity source and the battery source through an electrical circuit. In some embodiments, the electrode is implantable within the subject and at least one computer storage memory is in operable electrical communication remotely by a WiFi network or other remote network.

The disclosure relates to a computer program product encoded on a computer-readable storage medium comprising instructions for:
(a) measuring the mean arterial pressure (MAP) in a given cardiac cycle;
(b) measuring the heart rate (HR) in a given cardiac cycle;
(c) predicting a control cardiac response of the circulatory loop with at least a first control criterion over a control time period;
(d) calculating a desired adjustment value for MAP and/or HR to approach the control cardiac response;
(e) executing a signal command to stimulate the vagal nerve with an electrical pulse at a first, second and third location within a circulatory loop sufficient to adjust the MAP and/or HR in real-time with a magnitude corresponding to the desired adjustment value. In some embodiments, step (c) comprises: (i) applying a piece-wise linear or multiple local linear functions corresponding to interaction of MAP and HR within the circulatory loop. In some embodiments, step (c) further comprises: (ii) determining the probability of accomplishing the control cardiac response using a switch function. In some embodiments, step (d) comprises:
   (iii) calculating the weight of the step of predicting using the measured values of (a) and (b).

In some embodiments, step (d) comprises: (iv) calculating the total frequency of action potentials sufficient to adjust the MAP and/or HR in real-time with a magnitude corresponding to the desired adjustment value, wherein the total frequency of action potentials is based upon a modeled output value of step (c) and the real-time measured values of steps (a) and (b). In some embodiments, the computer program product disclosed herein further comprises: (f) repeating steps (a) through (e) over a set time period for continuous monitoring of HR and MAP.

In some embodiments, step (e) comprises: adjusting pulse amplitude and pulse frequency across the first, second and third locations of the circulatory loop to alter HR and MAP. In some embodiments, at least one of the first, second or third locations is along or proximate to a nerve fiber on the vagal nerve.

The disclosure relates to a computer program product operable in a system or device within a system that applies an algorithm to predict a control, or healthy, HR or MAP of a subject, that measures the real-time HR and MAP of the subject, calculates a desired adjustment value for the HR and MAP of the subject and delivers a command to an electrode embedded within the subject to stimulate the vagal nerve with an electrical pulse or series of electrical pulse that are of a magnitude equivalent to the desired adjustment value.

In some embodiments, the disclosure relates to a computer program product that comprises instruction for a piece-wise linear function for prediction of a control cardiac response, wherein the piece-wise linear function comprises:

$${x}^{i} ( k + 1 ) = {A}^{i} {x}^{i} ( k ) + {B}^{i} u ( k ) + {Bd}^{i} {d}^{i} ( k ) .$$

$${y}^{i} ( k ) = {C}^{i} {x}^{i} ( k ) + {D}^{i} u ( k ) + {d}^{i} ( k ) ,$$

wherein the superscript i represents the model number; di(k) is assumed Gaussian noise with zero mean imposed on the outputs, Aⁱ, Bⁱ, Cⁱ, Dⁱ are operating ranges of MAP in a cardiac cycle, k is the cardiac cycle number in which the numbers are being calculated, x is the operating region in cycle k, and y is the operating region in cycle k+1, u is an input value of MAP. In some embodiments, the desired adjustment value for the stimulation parameters in respect to MAP is calculated by formula:

$${min}_{\begin{matrix}u ( k + i \vert k ) \\ i = 1 , 2 , … , {N}_{c}\end{matrix}} \text{?} ( {‖ y ( k + i \vert k ) - \text{?} ( k + i \vert k ) ‖}_{Q}^{2} + \text{?} {‖ u ( k + i \vert k ) - u ( k + i - 1 \vert k ) ‖}_{R}^{2} + \text{?} ‖ u ( k + i \vert k ) - \text{?} ( k + i \vert k ) \text{?}$$

$$\text{?} \text{indicates text missing or illegible when filed}$$

wherein Nc is the number of cardiac cycles in a control horizon; wherein k+ilk is prediction into future cardiac cycle number time k+i based on the measurement at current sampling instance k; yA is the estimated output number, r is the set point, u_{b} is the baseline input of MAP; and wherein Q is the output weight matrix; R is the input weight matrix; and P is the integral action.

The disclosure also relates to a system comprising:
(i) the computer program product of any of claims 1 through 1o; and
(ii) a processor operable to execute programs; and/or
a memory associated with the processor.
   In some embodiments, the system further comprises: (i) a processor operable to execute programs; (ii) a memory associated with the processor; (iii) a database associated with and operably connected to said processor and said memory; (iv) a computer program product stored in the memory and executable by the processor, the program being operable for: (a) measuring a mean arterial pressure (MAP) in a given cardiac cycle within a circulatory loop of the subject; (b) measuring a heart rate (HR) in a given cardiac cycle within a circulatory loop; (c) predicting a control cardiac response of the circulatory loop with at least a first control criterion over a control time period; (d) calculating a desired adjustment value for MAP and/or HR to approach the control cardiac response; (e) executing a signal command to stimulate the vagal nerve with an electrical pulse sufficient to adjust the MAP and/or HR in real-time with a magnitude corresponding to the desired adjustment value at a first, second and third location within the vagal nerve; and (v) an implantable device comprising the computer program product and at least a first, second, and third electrode in operable electrical communication with the processor.
   In some embodiments, the computer program product is operable for step (c) by applying a piece-wise linear or multiple local linear functions corresponding to interaction of MAP and HR within the circulatory loop. In some embodiments, the computer program product is operable for step (c) by (ii) determining the probability of accomplishing the control cardiac response using a switch function. In some embodiments, the computer program product is operable for step (d) by (iii) calculating the weight of the step of predicting using the measured values of (a) and (b). In some embodiments, the system comprises the computer program product operable for step (d) by (iv) calculating the total frequency of action potentials sufficient to adjust the MAP and/or HR in real-time with a magnitude corresponding to the desired adjustment value, wherein the total frequency of action potentials is based upon a modeled output value of step (c) and the real-time measured values of steps (a) and (b). In some embodiments, the system comprises a computer program product is further operable for: (f) repeating steps (a) through (e) over a set time period for continuous monitoring of HR and MAP. In some embodiments, the computer program product is operable for step (e) by adjusting pulse amplitude and pulse frequency across the first, second and third locations of the circulatory loop.

In some embodiments, the system comprises an implantable device comprising a controller and an embodiment of the aforementioned computer program product.

The disclosure also relates to a method of modulating heart rate of a subject comprising:
(i) stimulating the vagal nerve by applying pulses of electricity to three locations of the vagal nerve by a device embedded in the subject;
wherein the device comprises:
   (a) the computer program product with instructions for:
(aa) measuring the mean arterial pressure (MAP) in a given cardiac cycle;
(bb) measuring the heart rate (HR) in a given cardiac cycle;
(cc) predicting a control cardiac response of the circulatory loop with at least a first control criterion over a control time period;
(dd) calculating a desired adjustment value for the stimulation parameters for the MAP and/or HR to approach the control cardiac response;
(ee) executing a signal command to stimulate the vagal nerve with an electrical pulse sufficient to adjust the MAP and/or HR in real-time with a magnitude corresponding to the desired adjustment value at a first, second and third location within a circulatory loop; and
   (b) a processor operable to execute programs; and
   (c) a memory associated with the processor.
   In some embodiments, the method comprises a device of the disclosure, wherein the device further comprises a first, second, and third electrode positioned at, along or proximate to the vagal nerve of the subject. In some embodiments, the electrodes are capable of stimulating a nerve fiber at or proximate to the electrode with a pulse of electricity, with an adjustable amplitude and frequency. In some embodiments, the computer software product comprises instructions for: adjusting the amplitude and frequency of an electrical pulse or plurality of electrical pulses and stimulating the vagal nerve of the subject with the electrical pulse or plurality of pulses that are of a magnitude equivalent to a desired adjustment value, thereby modulating the HR and/or MAP of the subject. In some embodiments, the method further comprises the step of monitoring the MAP of the subject prior to step (i). In some embodiments, the method further comprises the step of monitoring the HR of the subject prior to step (i). In some embodiments, one or a plurality of steps are repeated the step of stimulating is repeated to accomplish continuous maintenance of a desired HR and or MAP on a continuous basis. In some embodiments, the computer program product is programmable such that a time may be.

The disclosure relates to a method of modulating mean arterial pressure within the circulatory system of a subject comprising:
(i) stimulating the vagal nerve of the subject by applying pulses of electricity to three locations of the vagal nerve by a device embedded in the subject;
wherein the device comprises:
(a) any of the above-identified computer program products; and
(b) a processor operable to execute programs; and
(c) a memory associated with the processor.
   In some embodiments, the device further comprises a first, second, and third electrode positioned at or proximate to the vagal nerve of the subject. IN some embodiments, the method of modulating MAP further comprises one or more steps chosen from: monitoring the MAP of the subject prior to step (i); and/or monitoring the HR of the subject prior to step (i).

The disclosure relates to a method of treating abnormal heart rate in a subject in need thereof comprising:
(i) stimulating the vagal nerve of the subject by applying pulses of electricity to three locations of the vagal nerve by a device embedded in the subject;
wherein the device comprises:
(a) any of the above-identified computer program products; and
(b) a processor operable to execute programs; and
(c) a memory associated with the processor.
   In some embodiments, the device further comprises a first, second, and third electrode positioned at or proximate to the vagal nerve of the subject. In some embodiments, the method of modulating MAP further comprises one or more steps chosen from: monitoring the MAP of the subject prior to step (i); and/or monitoring the HR of the subject prior to step (i).

The disclosure also relates to a method of treating hypertension in a subject in need thereof comprising:
(i) stimulating the vagal nerve of the subject by applying pulses of electricity to at least three locations of the vagal nerve by a device embedded in the subject;
wherein the device comprises:
(a) any of the above-identified computer program products; and
(b) a processor operable to execute the instruction on the computer program product.
   In some embodiments, the device further comprises a first, second, and third electrode positioned at or proximate to the vagal nerve of the subject. In some embodiments, the method of modulating MAP further comprises one or more steps chosen from: monitoring the MAP of the subject prior to step (i); and/or monitoring the HR of the subject prior to step (i).
   In some embodiments, one or a plurality of the steps are repeated to accomplish continuous maintenance of a desired HR and or MAP in the subject.

The disclosure relates to a method of treating arrhythmia in a subject in need thereof comprising:
(i) stimulating the vagal nerve of the subject by applying pulses of electricity to at least three locations of the vagal nerve by a device embedded in the subject;
wherein the device comprises:
(a) any of the above-identified computer program products; and
(b) a processor operable to execute instructions of the computer program product.
   In some embodiments, the device further comprises a first, second, and third electrode positioned at or proximate to the vagal nerve of the subject. In some embodiments, the method of modulating MAP further comprises one or more steps chosen from: monitoring the MAP of the subject prior to step (i); and/or monitoring the HR of the subject prior to step (i).
   In some embodiments, one or a plurality of the steps are repeated to accomplish continuous maintenance of a desired HR and or MAP in the subject.

The disclosure relates to a method of evaluating the toxicity of an agent in a subject comprising:
(a) positioning any of the disclosed systems or devices at or proximate to the vagal nerve of the subject;
(b) exposing the subject to at least one agent;
(c) measuring HR and MAP of the subject; and
(d) correlating the HR and MAP of the subject with the toxicity of the agent, such that, if the HR and MAP are increased or decreased, the agent is characterized as toxic and, if the MAP and HR of the subject are unchanged, the agent is characterized as non-toxic;
wherein step (c) optionally comprises calculating the heart rate and/or mean arterial pressure of the subject; and
wherein step (d) optionally comprises correlating one or more of the heart rate or mean arterial pressure of the subject with the toxicity of the agent, such that, if the heart rate or mean arterial pressure of the subject decreased or increased, the agent is characterized as toxic or prone to toxicity and, if the heart rate or mean arterial pressure of the subject are unchanged, the agent is characterized as non-toxic.
   The disclosure relates to a method of evaluating the toxicity of an agent in a subject comprising:
(a) exposing the subject comprising any of the disclosed systems or devices to at least one agent;
(b) measuring HR and MAP of the subject using any of the disclosed systems or devices; and
(c) correlating the HR and MAP of the subject with the toxicity of the agent, such that, if the frequency and amplitude of pulse are increased or decreased, the agent is characterized as toxic and, if the frequency and amplitude of pulse of the subject are unchanged, the agent is characterized as non-toxic;
wherein step (b) optionally comprises calculating the heart rate and/or mean arterial pressure of the subject; and
wherein step (c) optionally comprises correlating one or more of the heart rate or mean arterial pressure of the subject with the toxicity of the agent, such that, if the heart rate or mean arterial pressure of the subject decreased or increased, the agent is characterized as toxic or prone to toxicity and, if the heart rate or mean arterial pressure of the subject are unchanged, the agent is characterized as non-toxic.
   In some embodiments, the at least one agent comprises a small chemical compound. In some embodiments, the at least one agent comprises at least one environmental or industrial pollutant.
   In some embodiments, the at least one agent comprises one or a combination of small chemical compounds chosen from: chemotherapeutics, analgesics, cardiovascular modulators, cholesterol level modulators, neuroprotectants, neuromodulators, immunomodulators, anti-inflammatories, and anti-microbial drugs.

The disclosure relates to a method of monitoring the heart rate or blood pressure of a subject comprising:
(a) positioning the system or device disclosed herein at or proximate to the vagal nerve of the subject;
(b) measuring HR and MAP of the subject using the system of device disclosed herein, such system or device comprising a sensor that is operably linked to the device and/or system and capable of measuring pulse amplitude or pulse frequency; and
(c) correlating the frequency and amplitude of pulse of the subject with the heart rate or blood pressure of the agent, such that, if the frequency and amplitude of pulse are increased or decreased, the heart rate or blood pressure is characterized as increased or decreased, respectively, and if the frequency and amplitude of pulse of the subject are unchanged, the heart rate or blood pressure is characterized as unchanged;
wherein step (c) optionally comprises calculating the heart rate and/or mean arterial pressure of the subject using a linear regression model.
   In some embodiments, the device is implantable within the subject. In some embodiments, steps (b) and (c) are accomplished using an implantable device comprising the computer program product disclosed herein.

### BRIEF DESCRIPTION OF DRAWINGS

FIG. 1 depicts a rat cardiac system model with a proposed circuit connecting graphical representations of sensors relative to graphical representation of the position of subject anatomy. A legend for the abbreviated components of the device and its components is as follows: P, pressure; V, volume; C, compliance; E, elastance; R, resistance; T, time period; f, frequency; I, amplitude; strain; lh, left heart; au, upper body arteries; al, lower body arteries; vu, upper body veins; vl, lower body veins; av, aortic valve; mv, mitral valve; su, peripheral resistance in upper body; sl, peripheral resistance in lower body; sa, arterial resistance; sv, venous resistance; as, afferent baroreceptive pathway; es, efferent sympathetic pathway; ev, stimulus.

FIGS. 2A and 2B depict PAWN global sensitivity analysis with respect to MAP (FIG. 2A) and HR (FIG. 2B). The parameter for each index is as follows: 1—Rsl2; 2—d,f es; 3—d,f ev; 4—GE; 5—GTs; 6—GVtot.

FIG. 3 depicts a block diagram representation of the MMPC algorithm, wherein each block represents a step in the algorithm logic to estimate a desired prediction of and adjustment to mean arterial blood pressure and heart rate of a subject wearing the disclosed device or system.

FIG. 4 depicts a block diagram for hardware-in-the-loop implementation of the system when embedded in a patient (on the right) operably communicating with a controller and simulation bank of data stored on a memory (on the left).

FIG. 5A through FIG. 5E depicts a set point tracking for MMPC. Comparison of MMPC shown for the output response. The parameters used in weight calculation are:]

$$\text{?} = [ \begin{matrix}\frac{\text{?} 1}{{MAP}_{b}} & 0 \\ 0 & \frac{0.1}{{HR}_{b}}\end{matrix} ]$$

$$\text{?}$$

$$\text{?} \text{indicates text missing or illegible when filed}$$

MAPb, are baseline MAP and HR in the nominal operating region.

FIG. 6A through 6B depict disturbance rejection by simulation of a +/−5%, +/−10%, +/−20% change in d_{fes}. The change occurs from cycle 10 to cycle 160. The open-loop output response with a 20% change in d_{fes} is shown by dotted line for comparison. The parameters used in weight calculation are:

$$Λ = {[ \begin{matrix}\frac{\text{?} 10}{{MAP}_{b}} & 0 \\ 0 & \frac{10}{{HR}_{b}}\end{matrix} ]}_{\text{?}}$$

$$\text{?} \text{indicates text missing or illegible when filed}$$

FIGS. 7A and 7B depict disturbance rejection by simulation of a +/−5%, +/−10%, +/−20% change in GTs. The change occurs from cycle 10 to cycle 160. The open-loop output response with a 20% change in GTs is shown by dotted line for comparison. The parameters used in weight calculation are:

$$Λ = {[ \begin{matrix}\frac{\text{?} 10}{{MAP}_{b}} & 0 \\ 0 & \frac{10}{{HR}_{b}}\end{matrix} ]}_{\text{?}}$$

$$\text{?} \text{indicates text missing or illegible when filed}$$

where MAPb and HRb are baseline MAP and HR in nominal operating region.

FIG. 8 depicts disturbance rejection by simulation of a change in d_{fes} and G_{Ts}. 10 percent decrease was made for both parameters from cycle 10 to 160. The open-loop output response is shown in red line and for comparison. The parameters used in weight calculation are:

$$Λ = {[ \begin{matrix}\frac{\text{?} 10}{{MAP}_{b}} & 0 \\ 0 & \frac{10}{{HR}_{b}}\end{matrix} ]}_{\text{?}}$$

$$\text{?} \text{indicates text missing or illegible when filed}$$

where MAP and HR in nominal operating region.

FIG. 9 depicts Event Adaptation for MMPC in MATLAB, with control decisions every 5 cardiac cycles. Exercise happens from 50 to 150 seconds. The parameters used in weight calculation are: The parameters used in weight calculation are:

$$Λ = [ \begin{matrix}\frac{100}{{MAP}_{b}} & 0 \text{?} \\ 0 & \frac{10}{{HR}_{b}}\end{matrix} ] ,$$

$$\text{?} \text{indicates text missing or illegible when filed}$$

where MAP and HR in nominal operating region.

FIG. 10 depicts an event adaptation for MMPC using HIL implementation, with control decisions every 5 cardiac cycles. All other conditions are the same as in FIG. 9.

FIG. 11 depicts a cardiovascular system model.

FIG. 12 depicts a block diagram for a baroreflex system.

FIG. 13 depicts a schematic of the VNS device model. Left: visualization of the VNS interface. Right: block diagram.

FIG. 14 depicts a block diagram of the closed-loop VNS system.

FIG. 15 depicts a prediction of reduced (dashed) and full (solid) models for rest (upper) and exercise (lower) regime.

FIG. 16 depicts a hemodynamic change from healthy to diseased state. SV: stroke volume, SAP: systolic arterial pressure, EF: ejection fraction, EDV(P): end diastolic volume(pressure).

FIG. 17 depicts a graph of P-V loops for nominal (black), hypertension (red), and VNS conditions (blue) for rest state.

FIG. 18 depicts dynamic response to exercise—nominal rat. MAP, AP ((mean)arterial pressure, mmHg), HR (heart rate, bpm), SVRL (systemic vascular resistance in lower body, mmHg/μL/s), Emax (max. left ventricular contractility, mmHg/μL).

FIG. 19 depicts hemodynamic changes from rest to exercise using the implant device and passing measured results through the disclosed algorithm: nominal (left), spontaneous hypertensive rat (right). CO: cardiac output.

FIG. 20 depicts a graph of P-V loops for nominal (black), hypertension (red), and VNS conditions (blue) for exercise state.

FIG. 21 depicts a graph of response of MAP and HR in percent with stimulation frequency and amplitude for a baroreceptive location.

FIG. 22 depicts a graph of response of MAP and HR in percent with stimulation frequency and amplitude for a non-baroreceptive location.

FIG. 23 depicts a graph of control results for nominal HR and MAP tracking in rest state. Measured outputs (left panel), estimated amplitude and frequency in baroreceptive, nonbaroreceptive, and vagal locations (right panel).

FIG. 24 depicts a graph of control results for nominal HR and MAP tracking in exercise state. Measured outputs (left panel), estimated amplitude and frequency in baroreceptive, nonbaroreceptive, and vagal locations (right panel).

FIG. 25 depicts a graph of output response for ten rats using NMPC with disturbance estimation. Mean (solid dark grey), max/min (dotted light grey), and mean±standard deviation (dashed grey).

FIG. 26 depicts a graph of control results for disturbance rejection. Measured outputs (left panel), estimated amplitude and frequency in baroreceptive, nonbaroreceptive, and vagal locations (right panel). NMPC: controller without disturbance estimation; NM-PCD: controller with disturbance estimation. The open-loop output response is shown in dotted line for comparison.

FIG. 27 depicts a graph of control results for adapting scenarios by switching model. Measured (dark grey) and setpoint (grey) of output response (left panel), estimated amplitude and frequency in baroreceptive, nonbaroreceptive, and vagal locations (right panel).

FIG. 28 depicts a graph of control results for adapting scenarios by estimating disturbance. Measured (dark grey) and desired trajectory (grey) of output response (left panel), estimated amplitude and frequency in baroreceptive, nonbaroreceptive, and vagal locations (right panel).

### DETAILED DESCRIPTION OF EMBODIMENTS

### Definitions

Various terms relating to the methods and other aspects of the present disclosure are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “more than 2” as used herein is defined as any whole integer greater than the number two, e.g. 3, 4, or 5.

The term “about” as used herein when referring to a measurable value such as an amount, a temporal duration, and the like, is meant to encompass variations of ±20%, 110%, ±5%, 11%, ±0.9%, ±0.8%, ±0.7%, +0.6%, ±0.5%, ±0.4%, ±0.3%, ±0.2% or ±0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

The term “plurality” as used herein is defined as any amount or number greater or more than 1.

As used herein, “substantially equal” can be, for example, within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

The term “cardiomodulatory” refers to a substance that has a modulatory effect on the circulatory system of a subject. Such substances can be readily identified using standard assays which indicate various aspects of cardiac activation, stimulation or depression, such as measuring electrophysiological activity of the heart muscle during an exposure to the substance.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a subject or patient's health status.

As used herein, the term “goodness of fit” or “GOF” refers to a test that is used to test if sample data fits a distribution from a certain population (i.e. a population with a normal distribution or one with a Weibull distribution). In some embodiments, the GOF score of the disclosure can be calculated as described in Example 2.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof. In some embodiments, the subject in need thereof is a human seeking prevention of a cardiac disorder. In some embodiments, the subject in need thereof is a human diagnosed with cardiac disorder. In some embodiments, the subject in need thereof is a human seeking treatment for cardiac disorder. In some embodiments, the subject in need thereof is a human undergoing treatment for cardiac disorder.

As used herein the terms “electronic medium” mean any physical storage employing electronic technology for access, including a hard disk, ROM, EEPROM, RAM, flash memory, nonvolatile memory, or any substantially and functionally equivalent medium. In some embodiments, the software storage may be co-located with the processor implementing an embodiment of the invention, or at least a portion of the software storage may be remotely located but accessible when needed.

The term “electrical stimulation” refers to a process in which the cells are being exposed to an electrical current of either alternating current (AC) or direct current (DC). The current may be introduced into the solid substrate or applied via electrodes or other suitable components of the implantable system. In some embodiments, the electrical stimulation is provided to the device or system by positioning one or a plurality of electrodes at different positions within the device or system to create a voltage potential across subject's nerve fibers. The electrodes are in operable connection with one or a plurality of amplifiers, voltmeters, ammeters, and/or electrochemical systems (such as batteries or electrical generators) by one or a plurality of wires. Such devices and wires create a circuit through which an electrical current is produced and by which an electrical potential is produced across the device or system.

The term “plastic” refers to biocompatible polymers comprising hydrocarbons. In some embodiments, the plastic is selected from the group consisting of: Polystyrene (PS), Poly acrylo nitrile (PAN), Poly carbonate (PC), polyvinylpyrrolidone, polybutadiene (PVP), Polyvinyl butyral (PVB), Poly vinyl chloride (PVC), Poly vinyl methyl ether (PVME), poly lactic-co-glycolic acid (PLGA), poly(l-lactic acid), polyester, polycaprolactone (PCL), poly ethylene oxide (PEO), polyaniline (PANI), polyflourenes, polypyrroles (PPY), poly ethylene dioxythiophene (PEDOT), and a mixture of two or any two or more of the foregoing polymers. In some embodiments, the plastic is a mixture of three, four, five, six, seven, eight or more polymers.

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any non-human mammal. The present disclosure relates to any of the methods or compositions of matter wherein the sample is taken from a mammal or non-human mammal. The present disclosure relates to any of the methods or compositions of matter wherein the sample is taken from a human or non-human primate.

As used herein, the term “predicting” refers to making a finding that an individual has a significantly enhanced probability or likelihood of experiencing a biological response or event. In some embodiments, predicting means making a finding that an individual has a significantly enhanced probability or likelihood of benefiting from and/or responding to an cardiac treatment. In some embodiments, the cardiac treatment is administration of an HR modulating agent. In some embodiments, the cardiac treatment is administration of a MAP-modulating agent. In some embodiments, the cardiac treatment is administration of a beta-blocker, vasodilator or vasoconstrictor. In some embodiments, the cardiac treatment is a therapy capable of modifying the effects of arrhythmia, abnormal heart rate or abnormal blood pressure.

A “score” is a numerical value that may be assigned or generated after normalization of the value based upon the presence, absence, or value of HR, MAP and/or blood pressure parameters, such as amplitude or frequency of blood pressure stimuli within a subject. In some embodiments, the score is normalized in respect to a control data value.

As used herein, the term “stratifying” refers to sorting individuals into different classes or strata based on the features of a cardiac disorder. For example, stratifying a population of individuals with heart disease involves assigning the individuals on the basis of the severity of the disease (e.g., mild, moderate, advanced, etc.).

As used herein, the term “subject,” “individual” or “patient,” used interchangeably, means any animal, including mammals, such as mice, rats, other rodents, rabbits, dogs, cats, swine, cattle, sheep, horses, or primates, such as humans. In some embodiments, the subject is a human seeking treatment for a cardiac disorder. In some embodiments, the subject is a human diagnosed with cardiac disease. In some embodiments, the subject is a human suspected of having a cardiac disorder. In some embodiments, the subject is a healthy human being.

As used herein, the term “threshold” refers to a defined value by which a normalized score can be categorized. By comparing to a preset threshold, a subject, with corresponding qualitative and/or quantitative data corresponding to a normalized score, can be classified based upon whether it is above or below the preset threshold.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

The terms “significantly enhanced” means that the numbers an observed enhancement within a set of data is unlikely to have happened by chance, normally identified as a p value.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of a cardiac disorder. The activity contemplated by the present methods includes both medical therapeutic and/or prophylactic treatment, as appropriate. The specific dose of a compound or agent administered according to the present disclosure to obtain therapeutic and/or prophylactic effects will, of course, be determined by the particular circumstances surrounding the case, including, for example, the compound administered, the route of administration, and the condition being treated. In some embodiments, the effective amount is an effective amount of electrical stimulation at the vagal nerve of a subject measured by amplitude and frequency of an electrical pulse. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue.

The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores of a size and shape sufficient to allow diffusion or non-active transport of proteins, nutrients, and gas through the solid substrate. In some embodiments, devices of the disclosure comprise a housing comprising a solid substrate the comprises two three or more exterior surfaces and two, three or more interior surfaces defining an interior space within which at least one circuit is positioned, the circuit operably connecting electrical communication among at least a first sensor, from about 1 to about 4 electrodes, a controller comprising any disclosed computer program product, and a computer storage memory. In some embodiments, an electrode, or two electrodes or at least three electrodes are positioned on the exterior surface of the housing in three different positions, such that after implantation within a subject, the electrodes are positioned on three discrete locations along or proximate to the vagal nerve fibers of the subject. In some embodiments, the device of the disclosure comprises one of a plurality of pores that facilitate transport of biological fluid across the surface of the implantable device. One of ordinary skill could determine how big of a pore size is necessary based upon the contents of the device, the amount of exposure to the solid substrate or its contents in a particular microenvironment. In some embodiments, the solid substrate is made of a biocompatible material. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that the interior surface of the base and define a cylindrical or substantially cylindrical interior chamber; and wherein an opening is positioned at one end of the chamber to allow an electrical connection, such as a wire to operably connect an electrode on the exterior surface to the electrical circuit contained within the chamber. In some embodiments, the base comprises one or a plurality of pores of a size and shape sufficient to allow diffusion of protein, nutrients, and oxygen through the solid substrate. In some embodiments, where the solid substrate comprises a hollow interior portion defined by at least one interior surface, the cells in suspension or tissue explants may be seeded by placement of cells at or proximate to the opening such that the cells may adhere to at least a portion the interior surface of the solid substrate for prior to growth. The at least one compartment or hollow interior of the solid substrate allows a containment of the circuit of the device and limits exposure to the subject's immune system. In some embodiments, the solid substrate is cylindrical, tubular or substantially tubular or cylindrical such that the interior compartment is cylindrical or partially cylindrical in shape. In some embodiments, the solid substrate comprises one or a plurality of branched tubular interior compartments. In some embodiments, the bifurcating or multiply bifurcating shape of the hollow interior portion of the solids is configured for or allows sensors and/or electrodes to be positioned in multiple branched patterns on or proximate to the exterior of the device. When and if electrodes are placed at to near the distal end of a subject's nerve fiber and at or proximate to a neuronal cell soma, electrophysiological metrics, such as intracellular action potential can be measured and/or delivered by the device or system comprising the device. In some embodiments, the electrodes are operably linked to a voltmeter, ammeter and/or a device capable of generating a current on a length of wire physically connecting the electrodes to the voltmeter, ammeter and/or device.

The disclosure relates to a device capable of both sensing HR values and MAP values in a subject, then stimulate a user with an electrical pulse comprising one or more stimulation parameters. In some embodiments, the device is capable of sensing intrinsic depolarizations. Depolarizations are represented by the P-wave (atrial lead) and QRS complex (ventricular lead). T-waves reflect repolarization and should not be sensed by a device stimulating the vagal nerve. Sensing is used to inhibiting or triggering pacing pulses. The inhibition of pacing is appropriate when there is intrinsic cardiac activity; the presence of spontaneous atrial or ventricular activity should inhibit pacing in the chamber with activity. However, sensing of spontaneous atrial activity (P-waves) without subsequent ventricular activity (QRS) should result in stimulating the vagal nerve. In some embodiments, to sense correctly, the device must detect near-field depolarization currents (P or QRS), and ignore near-field repolarization currents (T-waves), as well as far-field currents (i.e. currents generated by tissues that the electrode is not connect to). Also, external signals from electronics (cell phones, computers, etc) must also be ignored. The atrial lead is therefore set to record signals with an amplitude range of from about 1.5 to about 5 mV, and frequency of about 80 to about 100 Hz. The ventricular lead records signals in a range from about to to about 30-Hz range and from about 5 to about 25 mV in amplitude.

The device may have one or more sensors. Cardiac sensors are known in the art and one or more implantable devices of the disclosure comprise a sensor to detect heart rate and/or a sensor to detect arterial blood pressure of the subject. Heart rate sensors and arterial blood pressure sensors are disclosed in as non-limiting examples in WO2018045595 and WO2016040264, each of which is incorporated by reference in its entirety.

### Systems

The above-described methods can be implemented in any of numerous ways. For example, the embodiments may be implemented using a computer program product (i.e. software), hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device implantable within the subject. Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow a user, a subject or a physician treating the subject to make manual adjustments, make selections, enter data or various other information or parameters, and/or interact in any of a variety of manners with the processor during execution of the instructions. In some embodiments, the parameters include time period assessment for monitoring the cardiac cycle or cycles of the subject, elasticity of the circulatory system of the subject, cardiac control values, or any parameter identified in Tables I, II, or III.

The various methods or processes outlined herein may be coded as software that is executable on one or more processors that employ any one of a variety of operating systems or platforms. The disclosure also relates to a as a computer readable storage medium comprising executable instructions to perform any Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction and communicates the steps to a controller contained within an implantable device, the device implanted at or proximate to the vagal nerve of the subject.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor but may be distributed in a modular fashion amongst a number of different computers or processors to implement various aspects of the present invention. Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments. Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the disclosure relates to various embodiments in which one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In some embodiments, the disclosure relates to a computer-implemented method of determining an abnormality in blood pressure, heart rate, or pulse frequency or pulse amplitude of a subject, the method comprising:
(a) measuring the mean arterial pressure (MAP) in a given cardiac cycle;
(b) measuring the heart rate (HR) in a given cardiac cycle;
(c) predicting a control cardiac response of the circulatory loop with at least a first control criterion over a control time period;
(d) calculating a desired adjustment value for MAP and/or HR to approach the control cardiac response; wherein the steps are performed by a user through a system comprising:
(x) the computer program product with instructions for executing the steps (a) through (d);
(y) a processor operable to execute programs; and
(z) a memory associated with the processor; and wherein, if the difference between the control cardiac response and the desired adjustment value is significantly large, then the subject is characterized as having an abnormality of at least one of: blood pressure, heart rate, or pulse frequency or pulse amplitude. In some embodiments, the method further comprises a step (e) of executing a signal command to stimulate the vagal nerve with an electrical pulse sufficient to adjust the MAP and/or HR in real-time with a magnitude corresponding to the desired adjustment value at a first, second and third location within a circulatory loop. IN some embodiments, the disclosed methods are accomplished only by way of stimulation of the vagal nerve.

In some embodiments, the disclosure relates to a system that comprises at least one processor, a program storage, such as memory, for storing program code executable on the processor, and one or more input/output devices and/or interfaces, such as data communication and/or peripheral devices and/or interfaces. In some embodiments, the user device and computer system or systems are communicably connected by a data communication network, such as a Local Area Network (LAN), the Internet, or the like, which may also be connected to a number of other client and/or server computer systems. The user device and client and/or server computer systems may further include appropriate operating system software.

In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device disclosed herein.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method steps and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java™, BASIC, Pascal, Fortran, Cobol, assembly language, machine code, or the like.

Many of the functional units described in this specification have been labeled as circuits, in order to more particularly emphasize their implementation independence. For example, a circuit may be implemented as a hardware circuit comprising custom very-large-scale integration (VLSI) circuits or gate arrays, off-the-shelf semiconductors such as logic chips, transistors, or other discrete components. A circuit may also be implemented in programmable hardware devices such as field programmable gate arrays, programmable array logic, programmable logic devices or the like.

In some embodiment, the circuits may also be implemented in machine-readable medium for execution by various types of processors. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

The computer readable medium (also referred to herein as machine-readable media or machine-readable content) may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. As alluded to above, examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. As also alluded to above, computer readable program code embodied on a computer readable signal medium may be transmitted using any appropriate medium, including but not limited to wireless, wireline, optical fiber cable, Radio Frequency (RF), or the like, or any suitable combination of the foregoing. In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The computer readable program code may execute entirely on a user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

Functions, operations, components and/or features described herein with reference to one or more embodiments, may be combined with, or may be utilized in combination with, one or more other functions, operations, components and/or features described herein with reference to one or more other embodiments, or vice versa.

The disclosure relates to a computer program product integrated into or in electrical communication with a controller and a device disclosed herein. The device comprises at least one or two sensors, the sensor or sensors capable of measuring blood pressure, mean arterial pressure and/or heart rate of the subject. In some embodiments, the device further comprises an electrode operably connected to a wire and the controller such that variable parameters of electric current may be administered to a subject adjacent to or proximate to the sensor on the device. In a disclosed system, the device, controller and computer program product are operably connected by a circuit and commands from the computer program product are executed through the controller and device. Settings on the controller allow for adjustable magnitudes of amplitude and adjustable numbers of frequency of electrical pulses to be selected may be administered through the electrode. The computer program product is capable of measuring HR and MAP and calculating a desired adjustment value, that is the magnitude of selection parameters of both frequency and amplitude of electrical pulse sufficient to alter the heart rate and MAP of a user or subject to arrive or trend toward a control value. The control value, or optimal HR and MAP are considered the control cardiac response. Additional parameters of the system may be selected by a user to alter the duration of a particular setting. As an example a control cardiac response may have a control time or duration by which the control value may be achieved. IN some embodiments, the control value is from about 1 to about 100 mins in duration. In some embodiments, the control value is set to a continuous setting allowing an implantable device to operate continuously through several cycles.

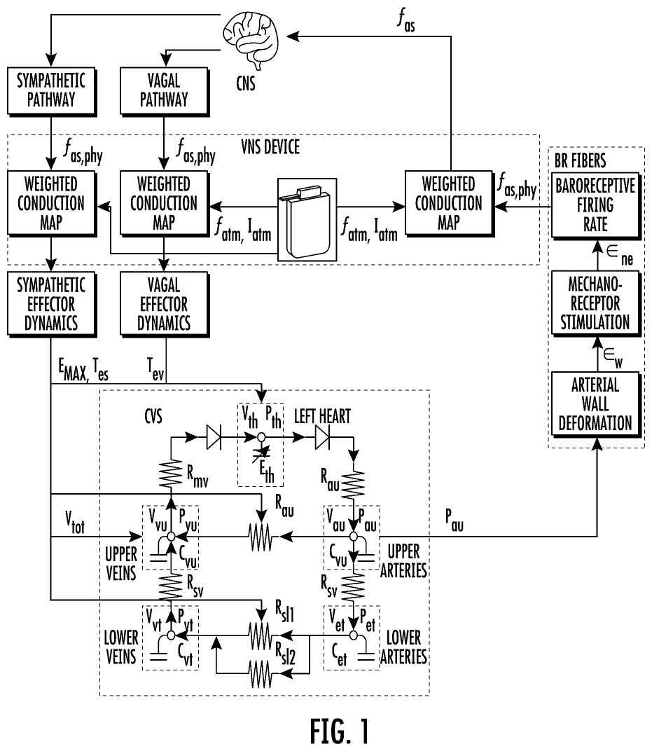

In some embodiments, the device is placed at or proximate to a component of the subject's circulatory system, or “circulatory loop” that comprises neuromuscular fibers of the vagal nerve and the circulatory system components upon which the vagal nerve acts. Administration of electrical pulses at or near this component of the subject induces a chance in the HR and MAP of the subject as depicted in FIG. 1. In some embodiments, the device is implanted at a positioned at or proximate to the vagal nerve such that stimulating electrodes on the device are positioned at least one, two or three different places on the vagal nerve fibers. Electrical pulses with magnitudes equivalent to the control criterion (control parameters) treat a subject with clinical deficiencies in HR and MAP.

In some embodiments, the device is implanted within a subject, such as a rodent or rat, such that the animal is an animal model for cardiac disorders. The disclosure relates to a system that can be used to screen a library of disease modifying agents such that HR and MAP may be measured in response to agents. Therefore, characterization of the agents as disease modifiers may be made as observation of improvement or no improvement of condition of the animal are monitored.

The disclosure relates to a system comprising a controller, optionally positioned within an implantable device, the controller operably and electrically linked to one or a plurality of sensors, a display, a charging chip, a Bluetooth communication device, and an electrode, each component in operable communication with a computer program product with instructions for executing steps:
(a) measuring the mean arterial pressure (MAP) in a given cardiac cycle;
(b) measuring the heart rate (HR) in a given cardiac cycle;
(c) predicting a control cardiac response of the circulatory loop with at least a first control criterion over a control time period;
(d) calculating a desired adjustment value of the stimulation parameters for MAP and/or HR to approach the control cardiac response;
(e) executing a signal command to stimulate the vagal nerve with an electrical pulse sufficient to adjust the MAP and/or HR in real-time with a magnitude corresponding to the desired adjustment value at a first, second and third location within a circulatory loop.
   In some embodiments, a device comprising one, two, three or more electrodes comprises the computer program product, the controller. In some embodiments, the device further comprises a clock, display, Bluetooth connector and a rechargeable battery source. In some embodiments, the device is implantable and the computer program product is operably connected to the device by a remote network, such as a Bluetooth network. In such cases, a software user, such a physician may input values for variable components of operation of the device remotely, and the device may still operate with those instructions. In some embodiments, the system comprises an implantable device capable of being stimulating the vagal nerve of a subject.

### Methods

Methods of the disclosure include a method of measuring or monitoring HR and MAP in a subject disclosed herein and methods of measuring toxicity or biological effect of a toxin, drug, therapeutic, biomolecule or pollutant when such molecules, drugs, or therapeutics are exposed to subject comprising the implantable device disclosed herein. The method may be computer-implemented whereby a server is in electrical communication with the device comprising at least one sensor capable of detecting pulse frequency and/or pulse amplitude, HR and/or MAP. IN some embodiments, the computer-implemented method relates to a system in which a controller positioned within the device implanted within the subject or remotely executes software commands to perform one or more of the following tasks: detect real time HR and MAP of a subject, predict a control cardiac response that is the control values of HR and MAP that are characterized as healthy for the subject, and stimulating an electrode within the device to stimulate the vagal nerve fibers of the subject with a frequency and amplitude sufficient to correct any difference between the control cardiac response values of a given cycle and the real-time measured values of the above identified parameters. In some embodiments, culture of spheroid and axons or neurites sprouting from such spheroid in the system. In some embodiments, the methods include a method of an implantable device stimulating the vagal nerve fibers of a subject to correct an abnormal HR an abnormal MAP or an abnormal pulse within the circulatory loop of the subject. In some embodiments, the system comprises a device comprising at least three electrodes that, when implanted, are position along or proximate to at least two or three different locations along the vagal nerve.

In some embodiments, any of the disclosed systems comprises an agent that stimulates, accelerates, slows or stops a cardiac abnormality in respect to pulse, HR or MAP of the subject. In some embodiments, any methods of the disclosure comprise stimulating only the vagal nerve to treat or prevent arrhythmia, or a cardiac disorder associated with abnormal blood pressure, or abnormal heart rate.

Methods of the disclosure relate to a method of treating a subject in need thereof with a pulse of electricity with a pulse amplitude and frequency sufficient to correct an abnormality in pulse, HR or MAP, wherein the pulse of electricity is administered directly to the vagal nerve in one, two or three distinct locations along the nerve or proximate to the nerve of the subject. In some embodiments, the methods are free of a step of stimulation of any other nerve fibers except those associated with the vagal nerve.

Methods of disclosure also relate to methods of screening a library of agents, such as agent known to or suspected of having cardiomodulatory effects. Electrophysiological studies of action potential generation can determine whether the observed animal model support predicted function, and the ability to measure clinically relevant endpoints or efficacy produces more predictive results of therapeutic potential of the agents. Similarly, information gathered from HR, MAP or other cardiac metrics of the subject can determine quantitative metrics for the degree of the overall cardiac health of the subject and lends further insight into toxic and neuroprotective, cardioprotective mechanisms of various agents or compounds of interest.

In some embodiments, the at least one agent comprises a small chemical compound. In some embodiments, the at least one agent comprises at least one environmental or industrial pollutant. In some embodiments, the at least one agent comprises one or a combination of small chemical compounds chosen from: chemotherapeutics, analgesics, cardiovascular modulators, cholesterol, neuroprotectants, neuromodulators, immunomodulators, anti-inflammatories, and anti-microbial drugs.

In some embodiments, the at least one agent comprises one or a combination of chemotherapeutics chosen from: Actinomycin, Alitretinoin, All-trans retinoic acid, Azacitidine, Azathioprine, Bexarotene, Bleomycin, Bortezomib, Capecitabine, Carboplatin, Chlorambucil, Cisplatin, Cyclophosphamide, Cytarabine, Dacarbazine(DTIC), Daunorubicin, Docetaxel, Doxifluridine, Doxorubicin, Epirubicin, Epothilone, Erlotinib, Etoposide, Fluorouracil, Gefitinib, Gemcitabine, Hydroxyurea, Idarubicin, Imatinib, Irinotecan, Mechlorethamine, Melphalan, Mercaptopurine, Methotrexate, Mitoxantrone, Nitrosoureas, Oxaliplatin, Paclitaxel, Pemetrexed, Romidepsin, Tafluposide, Temozolomide(Oral dacarbazine), Teniposide, Tioguanine (formerly Thioguanine), Topotecan, Tretinoin, Valrubicin, Vemurafenib, Vinblastine, Vincristine, Vindesine, Vinorelbine, Vismodegib, and Vorinostat.

In some embodiments, the at least one agent comprises one or a combination of analgesics chosen from: Paracetoamol, Non-steroidal anti-inflammatory drugs (NSAIDs), COX-2 inhibitors, opioids, flupirtine, tricyclic antidepressants, carbamaxepine, gabapentin, and pregabalin.

In some embodiments, the at least one agent comprises one or a combination of cardiovascular modulators chosen from: nepicastat, cholesterol, niacin, scutellaria, prenylamine, dehydroepiandrosterone, monatepil, esketamine, niguldipine, asenapine, atomoxetine, flunarizine, milnacipran, mexiletine, amphetamine, sodium thiopental, flavonoid, bretylium, oxazepam, and honokiol.

In some embodiments, the at least one agent comprises one or a combination of neuroprotectants and/or neuromodulators chosen from: tryptamine, galanin receptor 2, phenylalanine, phenethylamine, N-methylphenethylamine, adenosine, kyptorphin, substance P, 3-methoxytyramine, catecholamine, dopamine, GABA, calcium, acetylcholine, epinephrine, norepinephrine, and serotonin.

In some embodiments, the at least one agent comprises one or a combination of immunomodulators chosen from: clenolizimab, enoticumab, ligelizumab, simtuzumab, vatelizumab, parsatuzumab, Imgatuzumab, tregalizaumb, pateclizumab, namulumab, perakizumab, faralimomab, patritumab, atinumab, ublituximab, futuximab, and duligotumab.

In some embodiments, the at least one agent comprises one or a combination of anti-inflammatories chosen from: ibuprofen, aspirin, ketoprofen, sulindac, naproxen, etodolac, fenoprofen, diclofenac, flurbiprofen, ketorolac, piroxicam, indomethacin, mefenamic acid, meloxicam, nabumetone, oxaprozin, ketoprofen, famotidine, meclofenamate, tolmetin, and salsalate.

In some embodiments, the at least one agent comprises one or a combination of anti-microbials chosen from: antibacterials, antifungals, antivirals, antiparasitics, heat, radiation, and ozone.

The present disclosure additionally discloses a method of treating subject with a cardiac abnormality associated with heart rate, arterial blood pressure and/or heart failure by measuring parameters such as heart rate, arterial blood pressure of the subject and then administering a therapeutically effective electrical pulse to the vagal nerve of the subject. In some embodiments, the administration of an electrical pulse is accomplished through at least about 1, 2 or 3 or more electrodes positioned on the exterior of the disclosed device, wherein the electrodes are in operable communication with a circuit connecting the electrodes to a processor, controller and/or data storage memory. The magnitude of the therapeutically effective pulse is equal to a calculated desired adjustment value for parameters, frequency and amplitude of pulse, which is calculated as a function of the control cardiac response of the circulatory loop, which is a predicted value of the HR and MAP in a optimized system, and the real-time measurements of HR and MAP of a user/subject. In this way, for every cycle of a given cardiac output within a circulatory loop of a subject, the device is able to measure MAP and HR of the subject, predict a control value to either correct or maintain a healthy value for those metrics, and then calculate and execute a stimulate a stimulation parameter to correct or maintain the healthy HR and MAP in rela-time. In some embodiments, the stimulation parameters are only the amplitude and frequency of the electrical pulse being administered to the subject. Additional stimulation parameters may include duty cycle, ramp time, and duration of the pulse and any of these stimulation parameters may be independently selected manually by input on the system or controller operably connected to the computer program product. Alternatively, in some embodiments, the computer program product itself when operating in any of the disclosed systems may automatically calculate one or a plurality of stimulation parameters, thereby stimulating an electrical pulse through one, two or three different electrodes electrically and operably connected to the system components.

It is another object of the disclosure to provide a novel approach to evaluate cardiac physiology in vivo, using pulse frequency and amplitude as a correlative component as a clinically analogous metric to obtain real-time HR and MAP values for a subject in a given cardiac cycle that are more sensitive and predictive of human physiology than those offered by current methods.

It is another object of this disclosure to provide microengineered cardiac tissue that mimics native anatomical and physiological features and that is susceptible to evaluation using high-throughput electrophysiological stimulation and recording methods.

It is another object of the present disclosure to provide methods of replicating, manipulating, modifying, and evaluating mechanisms underlying cardiac diseases and peripheral neuropathies.

It is another object of the present disclosure to allow medium to high-throughput assay of neuromodulation in human neural cells for the screening of pharmacological and/or toxicological activities of chemical and biological agents that affect the circulatory system of a subject.

It is another object of the present disclosure to employ unique assembly of technologies such as 2D and 3D microengineered cardiac tissue in conjunction with optical and electrochemical stimulation and recording of human cardiac cells in vivo.

One embodiment provides an externally-controllable vagus nerve neurostimulator for treating chronic cardiac dysfunction. An implantable neurostimulator includes a pulse generator configured to drive electrical therapeutic stimulation tuned to restore autonomic balance through electrical pulses continuously and periodically delivered in both afferent and efferent directions of the cervical vagus nerve through a pair of electrodes via an electrically coupled nerve stimulation therapy lead. A programmable switch is configured to control the pulse generator in response to a remotely-applied magnetic signal. In some embodiments, the electrodes are helical.

A further embodiment provides implantable device for treating chronic cardiac dysfunction. An implantable neurostimulator device includes a pulse generator configured to deliver both afferent and efferent therapeutic electrical stimulation to a cervical vagus nerve in continuous alternating cycles of stimuli application and stimuli inhibition. A cervical vagus nerve stimulation therapy lead is electrically coupled to the pulse generator and is terminated by at least three electrodes through which the therapeutic electrical stimulation is delivered to the cervical vagus nerve at three different locations. A programmable switch configured to control the therapeutic electrical stimulation via the pulse generator in response to an external magnetic signal. IN some embodiments, the external magnetic signal is triggered by an abnormality detected in HR or MAP as calculated by the difference between a cardiac control response and a real-time measurement of HR or MAP within the subject.

A further embodiment provides an implantable device for facilitating control of electrical stimulation of cervical vagus nerves for treatment of chronic cardiac dysfunction. A cervical vagus nerve stimulation therapy lead includes electrodes configured to conform to an outer diameter of a cervical vagus nerve sheath of a patient and a set of connector pins electrically connected to the electrodes by an insulated electrical lead body. A neurostimulator can be powered by a primary battery and enclosed in a hermetically sealed housing. The neurostimulator includes an electrical receptacle included on an outer surface of the housing into which the connector pins are securely and electrically coupled. The neurostimulator also includes a pulse generator configured to therapeutically stimulate the cervical vagus nerve through the electrodes in alternating cycles of stimuli application and stimuli inhibition that are tuned to both efferently activate the heart's intrinsic nervous system and afferently activate the patient's central reflexes by triggering bi-directional action potentials. Finally, the neurostimulator includes a programmable switch configured to alter the triggering of the bidirectional action potentials by the pulse generator in response to a magnetic signal received from outside the housing.

A further embodiment provides a vagus nerve neurostimulator with autotitration for treating chronic cardiac dysfunction. An implantable neurostimulator includes a pulse generator configured to drive electrical therapeutic stimulation tuned to restore autonomic balance through electrical pulses continuously and periodically delivered in both afferent and efferent directions of the cervical vagus nerve through one, two or about three electrodes via an electrically coupled nerve stimulation therapy lead. A programmable switch is configured to trigger automatic titration of the electrical therapeutic stimulation progressively over a fixed period of time in response to a remotely-applied magnetic signal.

A further embodiment provides implantable device with autotitration for treating chronic cardiac dysfunction. An implantable neurostimulator device includes a pulse generator configured to deliver both afferent and efferent therapeutic electrical stimulation to a cervical vagus nerve in continuous alternating cycles of stimuli application and stimuli inhibition. A cervical vagus nerve stimulation therapy lead is electrically coupled to the pulse generator and is terminated by a pair of helical electrodes through which the therapeutic electrical stimulation is delivered to the cervical vagus nerve. A programmable switch is configured to trigger automatic titration of the therapeutic electrical stimulation progressively over a fixed period of time in response to an external magnetic signal.

A still further embodiment provides an implantable device for triggering autotitration of electrical stimulation of cervical vagus nerves for treatment of chronic cardiac dysfunction. A cervical vagus nerve stimulation therapy lead includes a pair of helical electrodes configured to conform to an outer diameter of a cervical vagus nerve sheath of a patient, and a set of connector pins electrically connected to the helical electrodes by an insulated electrical lead body. A neurostimulator is powered by a primary battery and enclosed in a hermetically sealed housing. The neurostimulator includes an electrical receptacle included on an outer surface of the housing into which the connector pins are securely and electrically coupled. The neurostimulator also includes a pulse generator configured to therapeutically stimulate the cervical vagus nerve through electrodes in alternating cycles of stimuli application and stimuli inhibition that are tuned to both efferently activate the heart's intrinsic nervous system and afferently activate the patient's central reflexes by triggering bi-directional action potentials. Finally, the neurostimulator includes a programmable switch configured to trigger automatic titration of the bi-directional action potentials into a predetermined set of stimulation parameters progressively over a fixed period of time in response to a magnetic signal received from outside the housing.

By restoring autonomic balance, therapeutic VNS operates acutely to decrease heart rate, increase heart rate variability and coronary flow, reduce cardiac workload through vasodilation, and improve left ventricular relaxation. Over the long term, VNS provides the chronic benefits of decreased negative cytokine production, increased baroreflex sensitivity, increased respiratory gas exchange efficiency, favorable gene expression, renin-angiotensin-aldosterone system down-regulation, and anti-arrhythmic, anti-apoptotic, and ectopy-reducing anti-inflammatory effects.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure. All referenced journal articles, patents, and other publications are incorporated by reference herein in their entireties.

### EXAMPLES

### Example 1—System and Software Model

Cardiovascular diseases are the leading cause of death globally over the last 15 years. The high morbidity and mortality therapeutics and the need for innovative solutions. Vagal nerve stimulation (VNS) is a FDA-approved therapy for treating epilepsy and treatment-resistant depression. Experimental and clinical evidence has demonstrated the physiological effects and clinical significance of VNS in disease, such as heart failure [1], arrhythmia [2], and hypertension [3]. There are many factors, such as the physiology of the vagal nerve, electrode design, and stimulation parameters, that influence the outcomes of VNS treatment of diseases. A standard VNS system consists of a cuff stimulation electrode attached around the left or right vagal nerve in the neck region, connected to a pulse generator implanted in the thoracic region. The stimulus is delivered to the vagal nerve with several adjustable parameters, such as the current amplitude, pulse width, pulse frequency, and duty cycle (“on-off” ratio) [4]. The range of these parameters is adapted from those used in the application of VNS to treat epilepsy and is adjusted based on patient perception during recurrent clinical visits. Currently, three multi-center open label studies (ANTHEM-HF [5], NECTAR-HF [6] and INOVATE-HF [7]) using VNS for treatment of heart failure have shown varying levels of clinical efficacy. This variable efficacy of VNS could result from different operating regimes for each trial, indicating the necessity to investigate an automatic closed-loop control method, enabling subject-specific optimal update of VNS parameters in real time. Several effective approaches have been reported for im plementing closed loop control of VNS to determine optimal stimulation parameters in animal studies. For example, standard proportional-integral controllers were designed to regulate heart rate of dogs [8], [9], pigs [10], and rats [11]. Another study used a model-based framework to tune the parameters of a proportional-integral controller before applying it on sheep to control heart rate [12]. The previously dis cussed controllers were designed as single-input-single-output systems. A more recent controller based on state-transition models was developed to manipulate multiple VNS parameters [13]. However, the accuracy of this controller is dependent on the number of states, which is limited by the memory of the implantable device. Our group previously developed a nonlinear model predictive control (NMPC) algorithm, which manipulates pulse frequency and pulse amplitude at multiple stimulation locations to control the heart rate and blood pressure simultaneously.

One of the challenges associated with this application of NMPC includes the development and validation of a predictive cardiac model to be used in NMPC. The variety of cardiac models in the literature, ranging from an individual cardiac myocyte to the whole circulatory system, make it difficult to decide which equations are accurate enough in capturing the cardiac dynamical response. It is also difficult to fit subject-specific parameters to these nonlinear equations. Another challenge involves the high computational cost of NMPC. The numerical complexity of NMPC prevents it from guaranteeing a global solution to the resulting nonlinear optimization problem within real-time requirements. Multiple model predictive control (MMPC) has been widely used for control of nonlinear systems to reduce the online computational burden in the fields of aerospace engineering chemical processing and biomedical systems The algorithm is based on linearization of the nonlinear system at multiple operating points, or alternately, experimental data-driven identification of local linear models at multiple operating points, and use of a weighted model bank as a prediction model. Employing multiple piecewise linear models of the nonlinear system significantly simplifies implementation of the controller and enables efficient global optimization of performance objectives.

In this Example, we propose a MMPC design procedure for closed-loop VNS, which is able to manipulate pulse amplitude and pulse frequency at three VNS locations to control heart rate (HR) and mean arterial pressure (MAP) in a previously developed physiological model of a rat. Synthetic data generated from the pulsatile cardiac model and the sub-space identification technique are used to identify multiple local linear models for the MMPC algorithm. This procedure mimics the eventual realistic development of multiple models from experimental data. The proposed controller design is tested with hardware-in-the-loop simulation studies using various disturbance and set point tracking case studies that constitute a pre-clinical assessment of the algorithm's safety and efficacy.

### A. Quantitative Rat Cardiac System

In order to synthesize and test the controller, we used a previously published rat cardiac model [21] as the “true” or “ground truth” model, to represent the effect of the two VNS parameters (pulse amplitude, mA and pulse frequency, Hz) applied at three stimulation locations on the two physiological variables (heart rate (HR) in bpm and mean arterial pressure (MAP) in mmHg). The structure of the rat cardiac model is illustrated in FIG. 1, which consists of the cardiovascular
main characteristics of the model from [21] are summarized next, and due to page limitations, the detailed equations are provided in the supplementary material, along with the associated MATLAB code.
1) Cardiovascular System: The cardiovascular system includes the left heart and four vascular compartments, as shown in the hydraulic analog or equivalent RC circuit of FIG. 1. The right heart and the pulmonary circulation are ignored in this model based on the assumption that they are healthy and not affected by VNS. The left heart is described by the series arrangement of a time-varying elastance. The elastance varies during the cardiac cycle as a consequence of the contractile activity of the ventricle. A linear combination of an exponential pressure/volume function and a linear pressure/volume function adapted from [22] is used to represent the pumping performance of the left heart. The vascular compartments are used to represent systemic circulation, differentiating among the upper and lower arteries (subscript au and al), and the lower and upper veins (subscript vu and vl). The pressure and volume in all compartments of the vascular system is described by enforcing conservation of mass at the capacities in the CVS segment in FIG. 1. The blood passes from the upper body veins to the left heart through the mitral valve, and the blood passes from the left heart to the upper body arteries through the aortic valve, mimicked as the ideal unidirectional valve with a constant resistance.

### Baroreflex System

The baroreflex system is described by four distinctive components: the afferent pathways, the efferent sympathetic pathways, the efferent vagal pathways, and the action of several effectors. The afferent pathway predicts the relationship between arterial pressure and the activity of the baroreflexive fibers. FIG. 1 predicts arterial wall deformation with blood pressure, Pau, as input and circumferential strain, w, as output. The mechanoreceptor stimulation model uses a second-order Voigt-body model to predict receptor deformation, ne, as a function of circumferential strain. The third system uses an integrate and fire model describing the BR firing rate, f_{as,phy}. by taking the receptor deformation, ne, as input. The model describing the other three components is derived from [24]. Increasing the firing rate of BR fibers results in a decrease in the frequency of the sympathetic fibers and an increase in the vagal tone. These are represented by using a monotonically decreasing exponential curve for the sympathetic activity and a monotonically increasing sigmoid curve for the vagal activity. The effectors responding to sympathetic drive are peripheral resistance (both in upper and lower body), maximum elastance of left heart (Eₘₐₓ), total unstressed blood volume (Vₜₒₜ). Both sympathetic and vagal activities influence the response of the heart period.

### VNS Device

The device model is used to predict the change in firing state induced by VNS parameters on three types of nerve fibers, representing BR fibers, sympathetic fibers and vagal fibers. The effect of the two stimulation parameters, namely, the pulse amplitude and frequency, are taken into consideration by the model. Stimulation of a particular amplitude has an influence on the likelihood of nerve fiber recruitment. An activation curve with a sigmoid function is used to predict the activation probability of each fiber involved in the VNS for specific pulse amplitude. Further, the total frequency of action potentials conducted to the somatic end of nerve fibers depends on the interaction between the externally induced frequency of stimulation and the physiologically induced frequency. As the frequency of the stimulus and/or the rate of the physiological input increases, conduction reliability decreases due to collisions between these two signals, as well as inter- and intra-signal loss of excitability during refractory periods. A linear fitting of a conduction map described in [25] is used to represent the change of firing rate of each recruited fiber as a function of intrinsic frequency and the external stimulation frequency. Finally, the fibers involved in VNS are distributed in different stimulation locations nonhomogenously, thus the firing rate of each type of fibers is calculate as the weighted sum of its firing rate based on the concentration in different locations.

### Hypertensive Cardiovascular Model

Hypertension is a leading risk factor in the development of cardiovascular diseases. Around 15%-18% of hypertensive patients have drug-resistant hypertension, making treatment and blood pressure control challenging with available therapies [26]. VNS has shown promising preclinical results, indicating it can be used as an alternative therapy for these scenarios.

Hypertension is a long-term condition in which the force of blood against the arteries is persistently elevated. The cause of hypertension is attributed to complex interaction between abnormality in heart, vessel and nervous system. The pathophysiology of hypertension remains unclear. Nevertheless, multiple hemodynamic changes, including increased peripheral resistance, reduced arterial compliance, a central shift in blood volume, increased sympathetic activities and de-creased vagal activities, are observed in common hypertensive cases [27]. Further, hypertension is usually accompanied with diastolic dysfunction, which is characterized by an obvious increase in the slope of the end-diastolic pressure-volume relation [28].

To investigate if a closed-loop VNS control design could alleviate these symptoms, we constructed a hypertensive model of the rat cardiovascular system by making specific changes
in the physiological parameters, described as diseased values in Table I. As expected, the hypertensive cardiovascular model exhibited a preserved ejection fraction, a higher blood pressure, a slightly higher heart rate, and a larger end-diastolic pressure, which matched the experimental data of spontaneously hypertensive rats reported in [28].

### Exercise Cardiovascular Model

Acute exercise triggers multiple hemodynamic and cardio-vascular responses, for example, an increase in cardiac output, which is primarily due to increase in heart rate, and to a lesser extent, due to augmentation of stroke volume. The enhanced cardiac output is redistributed with an increased blood flow to the active skeletal muscles in the lower body, which induces a remarkable metabolic vasodilation. Muscle vasodilation reduces the systemic vascular resistance, which results in a small increase in mean arterial pressure [29]. Exercise also induces parasympathetic withdrawal and sympathetic activation, which are a function of exercise intensity and the muscle mass recruited [30]. Furthermore, the exercise-induced hemodynamic changes are different in hypertensive and normotensive subjects. Studies have demonstrated that acute exercise significantly increased systolic blood pressure and heart rate in normal rats, but not in spontaneous hyper-tensive rats [31]. To demonstrate that our closed-loop VNS design can ac-count for exercise conditions, we constructed an example of exercise cardiovascular model of a hypertensive rat by modi-fying several physiological parameters, described as exercise values in Table I. Specifically, we separated the peripheral resistance in the lower body into two parallel resistors: Rₛₗ₁ representing the resting muscle resistance and Rₛₗ₂ represent-ing the active muscle resistance. We decreased the value of Rₛₗ₂ and introduced offset of frequency in sympathetic and vagal pathways. The gain of heart period, total stressed blood volume and systolic elastance are also modified to match the experimental data of spontaneous hypertensive rat during last minute of treadmill exercise in [32].

### Sensitivity Analysis

We conducted a sensitivity analysis to determine the relative contribution of each model parameter in Table I to variability in HR and MAP. Understanding the relative contribution of each disease- or exercise-related parameter to overall behavior of the controllable hemodynamic variables allowed us to prioritize these parameters to model disturbances due to inter-subject variability and postural change. These parameters were then used to demonstrate how our closed-loop VNS design could account for disturbance rejection.

Two main used in the sensitivity analysis of complex biological models are local- and global-sensitivity analysis. The local sensitivity analysis is derivative based, which considers uncertainty stemming from input variations around a specific point. This approach can be infeasible for complex models, where formulating the cost function is nontrivial, i.e., models with discontinuities do not always have well-defined derivatives in all domains of interest. The global sensitivity analysis considers variations of the inputs within their entire feasibility space. In this work, we performed a global sensitivity analysis because the model is discontinuous and the uncertainty of some cardiac- and neuronal-related parameters are difficult to investigate. We applied a density-based sensitivity analysis method called PAWN [33] to investigate the sensitivity of HR and MAP to the cardiac and neuronal parameters in Table I. Briefly, the PAWN approach uses cumulative distribution function (CDF) to characterize probability distribution of each output with random selection of parameters within their feasible space. The sensitivity of each parameter is measured by the distance between the unconditioned probability distribution of each output variable that is obtained when all parameters vary simultaneously, and the conditional probability distribution that is obtained when varying all parameters except the target parameter. The distance between the unconditional CDF and conditional CDF is determined by the Kolmogorov-Smirnov (KS) statistic for each fixed value of the target parameter. The sensitivity of the target parameter over all possible values is finally represented by PAWN index Tᵢ which is characterized as a statistic of KS (e.g., the mean or the median). The Tᵢ varies between 0 and 1. Tᵢ=O means the end, Tᵢ=1 on the output.

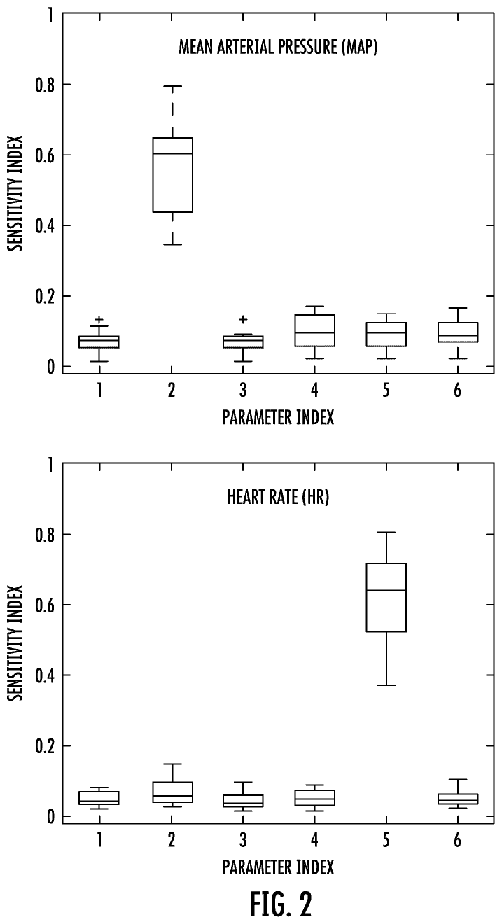

In this study, we performed 100 random samplings of the conditional CDFs for each parameter of interest within the range of diseased value and disease-exercise value. FIG. 2 illustrates six most influential parameters with respect to MAP. and HR. Results from PAWN sensitivity analysis show that the most influential parameter with respect to MAP is d_{fes} and the most influential parameter with respect to HR is G_{TS}. These functions test how the MMPC algorithm performs to reject these disturbances.

TABLE I

| | | | Nominal- | | Disease- |
|---|---|---|---|---|---|
| | | Nominal | Exercise | Diseased | Exercise |
| Parameters | Description | Value | Value | Value | Value |
| G_{E} | Gain of left heart | 0.475 | 0.95 | 0.95 | 1.425 |
| | systolic elastance | | | | |
| G_{Rsu} | Gain ot peripheral resistance | 0.0419 | 0.0419 | 0.1047 | 0.0419 |
| | in upper body | | | | |
| G_{Vtot} | Gain ot total stressed | 80 | 168 | 200 | 240 |
| | blood volume | | | | |
| A_{O} | Cross-section area of | 3.13 | 3.13 | 5 | 5 |
| | artery in relaxation state | | | | |
| d_{fev} | Offset of vagar flow | 0 | 0.5 | −3.2 | −2.7 |
| d_{fes} | Offset of sympathetic flow | 0 | 5 | 0 | 5 |
| O_{EIL} | Slope of end-diastolic pressure- | 0.006 | 0.006 | 0.0081 | 0.0081 |
| | volume relation | | | | |
| Rₛₗ₂ | Active muscle resistance | 100 | 0.1 | 100 | 0.1 |
| | in lower body | | | | |
| G_{Ts} | Gain of heart period in | −0.0045 | −0.0292 | −0.0082 | −0.0221 |

### MPC

Model predictive control (MPC) is a type of optimal control algorithm. Given a past trajectory of system inputs and outputs, the MPC algorithm uses a process model to predict the system response to a set of future control actions. At each sample time, a cost function that represents the error between the reference trajectory and predicted outputs is minimized over the prediction horizon to decide the optimal set of control actions over the control horizon. MPC implements the first control action, and the calculation is repeated at the next sample point, moving the prediction and control horizon forward by one step, when new measurements are available. The full cardiac model described in the preceding sections is computationally expensive and practically infeasible to be implemented as the predictor in MPC. On the other hand, a single local linear model cannot capture the nonlinear effects of the system. As such, a piece-wise linear or multiple local linear model functions as an alternative which has high prediction quality and low computational cost. The primary advantage of using this method is that it uses a common con-strained MPC formulation with a model bank and switching scheme to select an appropriate prediction model from this bank. Thus, it is computationally inexpensive to be solved in the desired sample period. In the following subsections, we introduce the design of piece-wise linear models using the sub-space identification technique and the MMPC algorithm. There are several methods reported in the literature for the model switching scheme, each with its own motivation and advantages. We use the switching scheme originally proposed in the context of Multi-Model Adaptive Control (MMAC) [19] and later applied in [20] using a probabilistic weighted prediction method.

### Model Bank

Since we use a rat cardiac model to represent the “true” system, a common approach to determine the piece-wise linear models is based on the Jacobian linearization of the full simulation model at each operating point. Here, we apply the sub-space identification technique using synthetic data generated from the “true” system model because: 1) the rat cardiac model is not continuously differentiable; 2) the proposed procedure mimics the eventual realistic development of multiple models from experimental data. The model bank is designed to encompass the entire anticipated MAP dynamics. The system state space is broken into i∈{1, . . . , 4}operating regions. The system dynamics are assumed to be locally linear in each operating region which covers 10 mm Hg MAP around its baseline point, defined by its output.

The four operating regions were defined by using the following procedure:
1) Select the baseline value of MAP in the current operating region (MAPi)_{b}.
2) Iteratively change the input u and simulate the system model until the MAP reaches MAPi at steady state; set the corresponding input as the corresponding input as u=uⁱ.
3) Determine the baseline value of HR in the current operating region (HRi) corresponding to u_{b}=uⁱb.
4) Define the current size of the operating region as MAPⁱb+/−10 mmHg.

The procedure is repeated for each operating region. The baseline values and operating regions are listed in Table II. The dynamics of each operating region are represented by a linear state space model that has the general form given as follows:

$${x}^{i} ( k + 1 ) = {A}^{i} {x}^{i} ( k ) + {B}^{i} u ( k ) + {Bd}^{i} {d}^{i} ( k ) .$$

$${y}^{i} ( k ) = {C}^{i} {x}^{i} ( k ) + {D}^{i} u ( k ) + {d}^{i} ( k )$$

where, the superscript i represents the model number; dⁱ(k) is assumed Gaussian noise with zero mean imposed on the outputs, which is used to account for uncertainty between the identified model and the full system being controlled. Subspace identification techniques and the Matlab implementation of the algorithm n4sid [34] were used to identify the piece-wise linear models from synthetic data generated by perturbing the “ground truth” model described below.
Several criteria are used to design input signals to generate synthetic data:
Each perturbation of input signal is chosen using a uniform distribution within the upper and lower bound, and is kept constant for five cycles to allow enough time for the system to respond.
A lower bound is chosen to ensure that a perturbation must be large enough to elicit a change in the outputs, and an upper bound is chosen to make sure a perturbation is not so large as to move the output response out of the defined region around the baseline point.

$$( 1 - α ) {u}_{b}^{i} \leq u \leq ( 1 + β ) {u}_{b}^{i} \qquad [ EQUATION 2 ]$$

where α and β are chosen according to the observed output response by manually manipulating the range of input signals.

TABLE II

| Model | Baseline Points | | | | | | | | Prediction Fit [%] | | Range |
|---|---|---|---|---|---|---|---|---|---|---|---|
| No. | MAPⁱ | HRⁱ | Ilⁱ | flⁱ | I2ⁱ | f2ⁱ | I3ⁱ | flⁱ | MAP | HR | of MAP |
| 1 | 96 | 310 | 1.1 | 45 | 0.1 | 1 | 1 | 45 | 87.22 | 91.13 | (, 105) |
| 2 | 116 | 380 | 1.15 | 46.36 | 0.35 | 5.36 | 0.33 | 6.93 | 86.25 | 74.34 | (105, 125) |
| 3 | 136 | 340 | 1 | 30 | 0.5 | 10 | 0.8 | 20 | 90.43 | 90.65 | (125, 145) |
| 4 | 155 | 373 | 0.8 | 20 | 0.5 | 10 | 0.5 | 10 | 86.97 | 83.31 | (145,) |

### MMPC Algorithm

The MMPC controller uses a weighted sum of the piecewise linear model bank and an optimizer to determine a set of VNS parameters that best meet the desired tracking of the output trajectory of HR and MAP. The primary advantage of MMPC is model adaptation according to the operating region and the ability to handle explicit input and output constraint

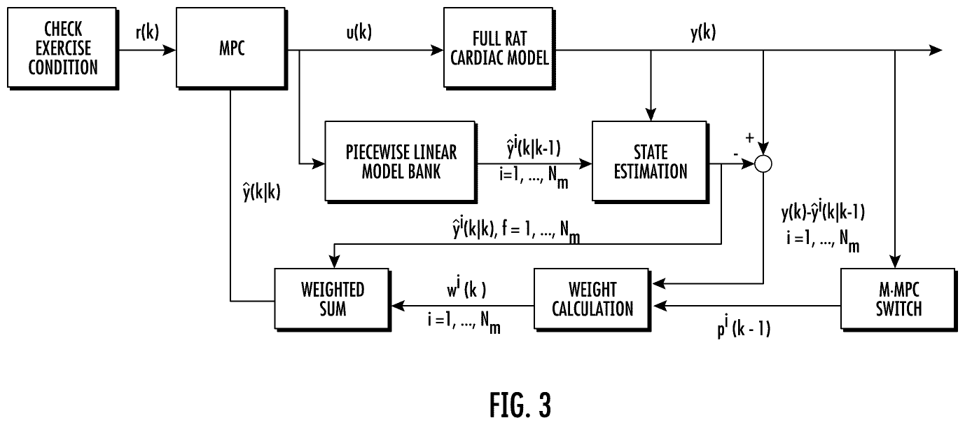

FIG. 3. The MMPC algorithm has the following steps:
1) At the current cardiac cycle k, the output measurement:
   (y(klk)) is fed to the state estimator, where a discrete Kalman observer estimates the current state (x\hat{ }Aⁱ(klk)) of each model, and provides an initial condition for future output estimation (y\hat{ }i(k+ilk),i∈(1, 2, . . . , Np)) over the prediction horizon Np. In this equation, x\hat{ } is a circumflex x and emant to be an estimated value of x; and y\hat{ } is a circumflex y and meant to refer to an estimated value for y.
2) A MMPC switch determines the previous probabilities of each model based on the current measurement of MAP. The residuals of output variables and previous probabilities are then used to calculate the weight of each model for prediction.
3) A convex quadratic optimization problem is solved in MPC, using the weighed sum of estimated outputs y\hat{ }ⁱ(klk) from each model, to give optimal control actions (u(k +ilk),i∈(1, 2, . . . , Nc)) over the control horizon. u(k+ilk)) is actuated by the VNS device. Then the procedure is repeated at the end of the cycle k+1. In this equation “y\hat{ }” is a circumflex y and meant to refer to an estimated value for y.

### MPC Formulation:

The MMPC uses the average of linear state space models in the model bank to predict future outputs. The MPC is formulated by solving the following quadratic objective function:

$${min}_{{u ( k + i \vert k )}_{i = 1 , 2 , … , {N}_{c}}} \text{?} ( {‖ \text{?} ( k + i \vert k ) - r ( k + i \vert k ) ‖}_{Q}^{2} + \text{?} ( ‖ u ( k + i \vert k ) - u ( k + i - 1 \vert k ) \text{?} + \text{?} ( ‖ u ( k + i \vert k ) - \text{?} ( k + i \vert k ) \text{?}$$

$$\text{?} \text{indicates text missing or illegible when filed}$$

estimated outputs, r is the set point, ub is the baseline input. The prediction horizon, Np =20, is chosen to be number of cardiac cycles for the open-loop system to reach its steady state; Nc=10 is the control horizon, which is chosen after extensive closed-loop tuning experiments. The output weight matrix Q penalizes deviation from the set point, with higher weighting on tracking the MAP setpoint to ensure that the physiological system remains close to the operating point weight matrix R penalizes the change of control actions. The third term in the objective function provides integral action—it penalizes deviations from baseline inputs and reduces oscillations.
Explicit constraints are imposed on input and output variables as follows:

$${u}_{min} \leq u ( k + i \vert k ) \leq {u}_{max} \qquad [ EQUATION 4 ]$$

$${y}_{min} \leq y ( k + i \vert k ) \leq {y}_{max}$$

The input constraints are chosen to meet biological safety constraints on stimulation intensity. A larger min-max range is designed for outputs, which functions as a soft constraint and allows a certain degree of violation on the desired range of HR and MAP.

### State Estimation:

Due to inter and intrasubject variability, there is always a degree of parameter uncertainty, change of system characteristics with time and disturbances affecting the system. To handle this situation, a disturbance model described in (1) is used for MPC with a Kalman filter framework for plant-model mismatch. An augmented state that combines the model states and disturbance terms, xⁱ(klk)=[xⁱ(klk), dⁱ(klk)]^{T}, is estimated by the observer. Using the augmented state, the disturbance model can be rewritten as follows:

$$[ EQUATIONS 5 AND 6 ]$$

$$[ \begin{matrix}{\hat{x}}^{i} ( k + 1 \vert k ) \\ {\hat{d}}^{i} ( k + 1 \vert k )\end{matrix} ] = [ \begin{matrix}{A}^{i} & {Bd}^{i} \\ 0 & I\end{matrix} ] [ \begin{matrix}{\hat{x}}^{i} ( k \vert k ) \\ {\hat{d}}^{i} ( k \vert k )\end{matrix} ] + [ \begin{matrix}{B}^{i} \\ 0\end{matrix} ] u ( k \vert k )$$

$${\hat{y}}^{i} ( k \vert k ) = [ \begin{matrix}{C}^{i} & I\end{matrix} ] [ \begin{matrix}{\hat{x}}^{i} ( k \vert k ) \\ {\hat{d}}^{i} ( k \vert k )\end{matrix} ] + {D}^{i} u ( k \vert k )$$

$${\hat{x}}_{a}^{i} ( k + 1 \vert k ) = {A}_{a}^{i} {\hat{x}}_{a}^{i} ( k \vert k ) + {B}_{a}^{i} u ( k \vert k )$$

$${\hat{y}}^{i} ( k \vert k ) = {C}_{a}^{i} {\hat{x}}_{a}^{i} ( k \vert k ) + {D}_{a}^{i} u ( k \vert k )$$

The Kalman observer is defined by the following equations:

$$[ EQUATION 7 ]$$

$${\hat{x}}_{a}^{i} ( k \vert k ) = {\hat{x}}_{a}^{i} ( k \vert k - 1 ) + {L}^{i} ( y ( k \vert k ) - {C}_{a}^{i} {\hat{x}}_{a}^{i} ( k \vert k - 1 ) - {D}_{a}^{i} u ( k \vert k - 1 ) )$$

$${\hat{y}}^{i} ( k \vert k ) = {C}_{a}^{i} {\hat{x}}_{a}^{i} ( k \vert k ) + {D}_{a}^{i} u ( k \vert k - 1 )$$

$${\hat{x}}_{a}^{i} ( k + 1 \vert k ) = {A}_{a}^{i} {\hat{x}}_{a}^{i} ( k \vert k ) + {B}_{a}^{i} u ( k \vert k )$$

The augmented state at current time k is estimated based on the difference between the current measurement from the system and the model prediction. Then the augmented model is used to predict the augmented state into the future. Li is the solution of the following discrete Riccati equation based on recursive updating for the covariance of arriving error.

$$[ EQUATIONS 8 , 9 , 10 ]$$

$$\text{?} {L}^{i} ( k ) \text{?} = {P}^{i} ( k \vert k - 1 ) {{C}^{{i}^{T}} ( {C}^{i} {P}^{i} ( k \vert k \text{?} - 1 ) {C}^{{i}^{T}} + {R}_{e}^{i} )}^{- 1}$$

$$P ( k \vert k ) = ( I - {L}^{i} ( k ) {C}^{i} ) {P}^{i} ( k \vert k - 1 )$$

$$P ( k + 1 \vert k ) = {A}^{i} P ( k \vert k ) {A}^{{i}^{T}} + {Q}_{e}^{i}$$

$$\text{?} \text{indicates text missing or illegible when filed}$$

Here, P (klk 1) is a priori covariance matrix for arriving error; P (klk) is a posteriori covariance matrix for arriving error; Ri is the covariance of the observation noise; Qⁱ is the
covariance of process noise.

Weight Calculation: Previously, four linear models were described to predict the system output within each local region Each linear model may provide a good description of local dynamics close to the baseline operating point, but both adjacent region models have a certain degree of probability to predict the output response when the system is close to a region boundary. Furthermore, an external disturbance entering use the probabilistic formulation of model switching with a weighted sum, originally proposed in [19], to predict the system output for controller design.

The probability for the ith model to be the predictive model at sampling time k is calculated using the following recursive

$${P}^{i} ( k ) = \frac{exp ( - 0.5 {{e}^{i} ( k )}^{T} Λ {e}^{i} ( k ) ) {P}^{i} ( k - 1 )}{{\sum }_{j = 1}^{{N}_{m}} exp ( - 0.5 {{e}^{j} ( k )}^{T} Λ {e}^{j} ( k ) ) {P}^{i} ( k - 1 )} \qquad [ EQUATION 11 ]$$

where, Nm is the number of models in the model bank,
eⁱ(k)=y(klk) yAⁱ(klk) is the ith model residual. The prediction error of each model forms a sequence of independent, Gaussian distributed random variables with zero mean, which results in the exponential term in (11). The tuning matrix, A, determines the speed of convergence of the probability. The probability calculation is recursive, depending on the probability in the previous step. In our controller design, a simple logic is that when a measurement of MAP is in current operating point has a higher probability of being the correct predictive model. Therefore, we manually update the previous probability to force the control to adapt to that model whenever an operating region switch occurs using the following equation:

$$\begin{matrix}[ Equation 12 ] & \\ {P}^{i} ( {k}^{*} ) = \{ \begin{matrix}p & M ( {k}^{*} ) = i \\ \frac{1 - p}{{N}_{m} - 1} & otherwise\end{matrix} & ( 12 )\end{matrix}$$

Each probability is bounded between zero and one, where larger value of Pi indicates greater probability that model i will accurately predict output response. M (k) is the expected model at the current operating conditions. k is the first step and the step where there is a switch in the operating region. An artificial limit, 5, is enforced to prevent the probability reaching zero since a zero probability would move the model out of the model bank in the future steps. The weight of each model is assigned by normalizing the probabilities as follows:

$${w}^{i} ( k ) = \{ \begin{matrix}\frac{{P}^{i} ( k )}{{\sum }_{j = 1}^{{N}_{m}} {P}^{j} ( k )} & {P}^{i} ( k ) > δ \\ 0 & {P}^{i} ( k ) = δ\end{matrix} \qquad [ EQUATION 13 ]$$

The resulting predicted output yA(klk), based on this weighted average prediction model is in the following form:

$$\hat{y} ( k \vert k ) = {\sum }_{j = 1}^{{N}_{m}} {w}^{j} ( k ) {\hat{y}}^{j} ( k \vert k ) \qquad [ EQUATION 14 ]$$

4) Switching Logic: The initial guess of probability of each model used in the MPC objective function is chosen based on the current operating condition. For a first step, the operating region and the expected model is determined by locating the online measurement of MAP in the MAP region described in Table II. After that, the operating region is updated based on the previous region (M (k 1)=i) and the current measurement of MAP using the following switching logic:

$$\begin{matrix}[ EQUATION 15 ] & \\ & ( 15 )\end{matrix}$$

$$M ( k ) = \{ \begin{matrix}i + 1 , & MAP ( k ) \geq {MAP}_{u}^{i} + γ \\ i , & {MAP}_{l}^{i} - γ < MAP ( k ) < {MAP}_{u}^{i} + γ \\ i - 1 , & MAP ( k ) \leq {MAP}_{l}^{i} - γ\end{matrix}$$

Iv. Hardware-In-the-Loop Experiment

Hardware-in-the-loop (HIL) simulation is a type of real-time simulation. It is used to validate the control algorithm by creating a virtual real-time environment. HIL is especially useful in the closed-loop design of medical devices prior to testing the control algorithm on animals or patients which tends to be expensive, time consuming and requires adherence to extensive safety protocols.

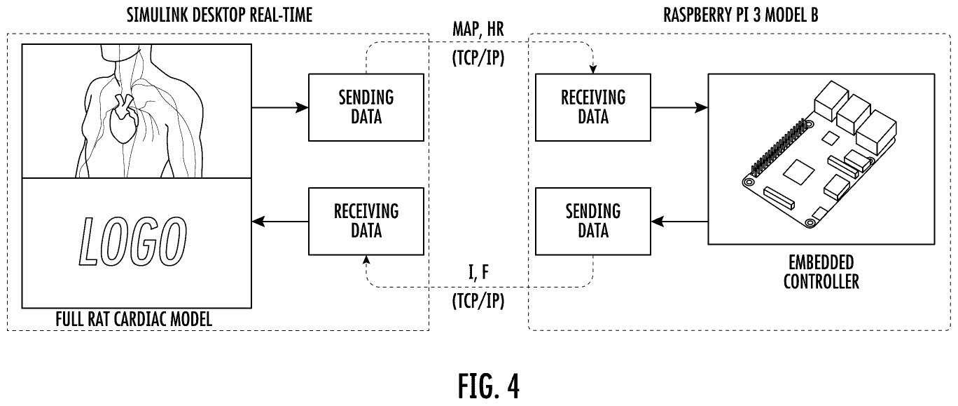

Our HIL implementation of the closed-loop control is illustrated in FIG. 4. The full rat cardiac model (“ground truth” model) was simulated in Simulink Desktop Real-Time using a fixed-step solver with the external mode. This simulation platform provides a real-time kernel for executing Simulink models on a laptop or desktop running Windows or MacOS. In external mode, the model and solver are converted into C code, built into a real-time executable, and run in a real-time kernel. The advantages of the external mode are the following: emulation of real-time performance; ability to connect to a range of ethernet or wireless I/O devices with supported library blocks; support for real-time tuning of simulation parameters; support for high sampling rates up to 20 KHz. The MMPC control action sequences, weight calculation, and the observer are deployed on a single-board computer known as Raspberry Pi 3 Model B (https://www.raspberrypi.org/). A 32 GB microSD card is used as the flash memory of the system as a 64-bit quad core ARM central processing unit (CPU) that operates up to 1.2 GHz clock speed. The Raspberry Pi emulates an embedded controller: it communicates with Simulink via TCP/IP connection to receive virtual MAP and HR data sent by the packed output block, and transmits the computed control action back to the packed input port in the Simulink Desktop Real-time wirelessly. All code for the controller in the Raspberry Pi is written in Python 3.8 and the quadratic programming problem inherent to the MMPC is solved using the CVXOPT toolbox [35].

### DISCUSSION

In this section, we present results to demonstrate how our control algorithm can regulate the HR and MAP with VNS. The performance of our control algorithm was evaluated for set point tracking, disturbance rejection and event triggered adaptation using Matlab implementation of the system model and the controller, where the control decision is made at the end of each cardiac cycle. After substantial MATLAB testing, the control algorithm was converted into python and implemented in Raspberry PI for HIL study to demonstrate its computational feasibility in an embedded controller. We demonstrate the results of set point tracking and disturbance rejection with the Matlab implementation, and HIL results are shown for the control of adaptation during rest to exercise scenarios.

### A. Set Point Tracking

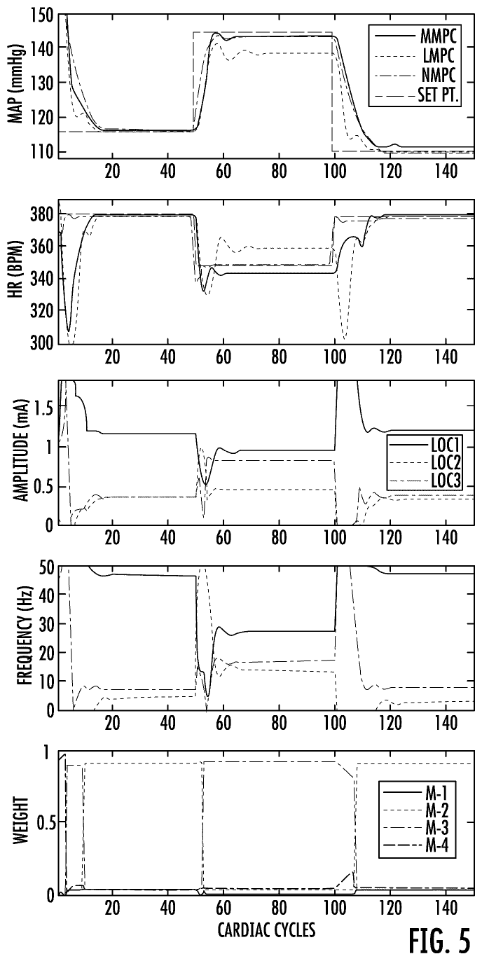

The MMPC for HR and MAP was evaluated by tracking three types of set points: baseline operating point, boundary of operating region, and non-baseline set point. The weight matrices and other parameters of MMPC were carefully tuned by extensive closed-loop simulation experiments. The set-point regulation results are illustrated in FIG. 5. For comparison, we
also present the output results using a local fixed model in MPC (LMPC) and using the NMPC presented in [21] for the same set-point changes. In this case, no disturbance was introduced and we did not estimate the augmented state in observer. This comparison is mainly presented to demonstrate the advantage of MMPC for controlling this complex nonlinear system.

The “ground truth” model was initialized at a steady-state MAP of 150 mmHg. The controller was switched on at k=0 with a baseline set point change to region two to decrease the MAP to 116 mmHg and decrease HR to 380 bpm. The second region was shown as a target in this simulation since this set point is obtained for modeling a healthy rat in rest state using the parameter sets described in Table I. Other operating regions show similar results. In this case, all of three control strategies (LMPC, MMPC, or NMPC) converge with zero offset in about 30 cardiac cycles, with NMPC showing smaller deviations for tracking of HR. The similarity between LMPC and MMPC is due to the fact that the dominant model used in MMPC converges to the nominal local model. But the difference in model weight based on the online measurements and model adaptation. Another setpoint change was made at k=50 to increase the MAP setpoint to 145 mmHg, with corresponding setpoint of HR computed to be 348 bpm, which corresponds to the boundary between regions three and four. The boundary tolerance works to minimize switching and prevents oscillatory dynamics at region boundaries. Clearly, the MMPC and NMPC performs better tracking results than LMPC. The improvement of MMPC can be explained by the involvement of model 3 and a large weight assigned to model 3 when the MAP increases from region two to region three, thereby reducing the controller convergence time. Finally, a non-baseline set point was introduced at k=100 to lower the MAP to 110 mmHg and to increase HR to 378 bpm. NMPC and MMPC perform better, but MPC with local model can deal with this non-baseline set point, indicating that this operating region is well defined to capture the dynamics of MAP using that nominal model.

The performance of MMPC depends on the tuning matrix in (11). Through extensive closed-loop simulations, we found small tuning weight may result in slow convergence, while a large tuning weight may converge to a different model and present oscillations on outputs. With current setting of A, MMPC converges to the local model quickly attributing to the initial guess of the model weight. Here, the average computational time per iteration for LMPC is 0.0102 seconds, for MMPC is 0.0115 seconds, and for NMPC is 1.7 seconds using MATLAB. The proposed MMPC algorithm can provide better tracking performance compared to LMPC and substantially reduces computational expense compared to NMPC.

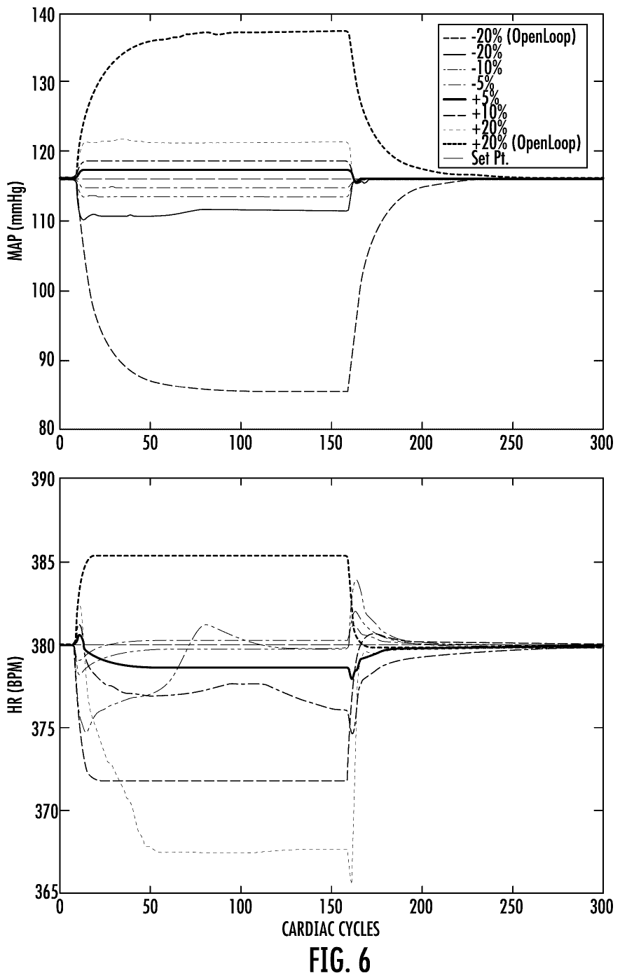

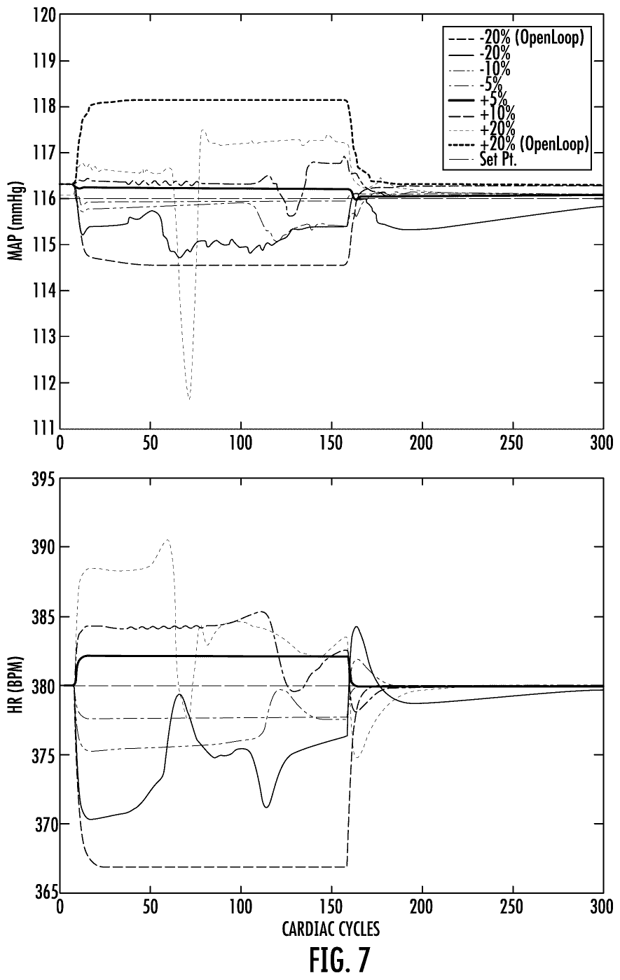

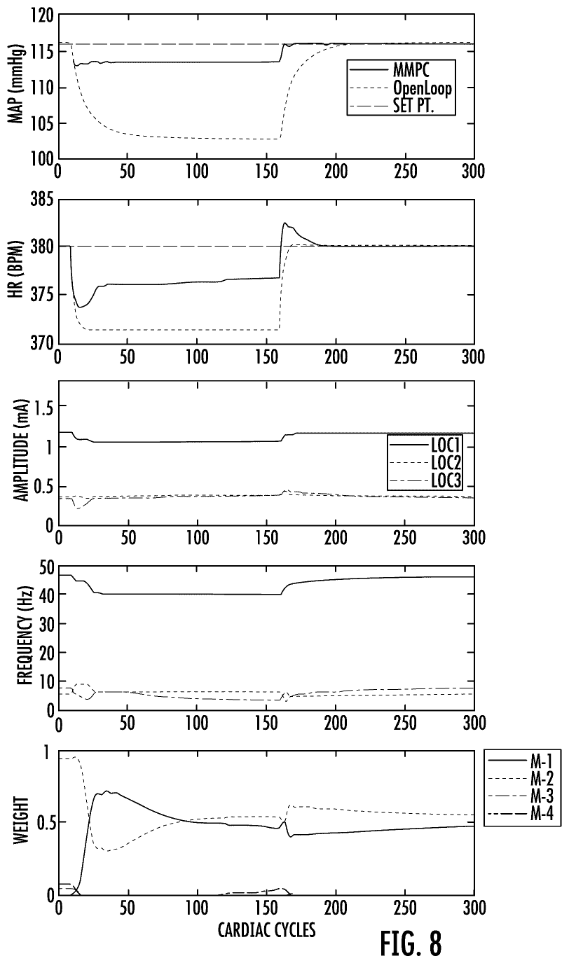

We test the MMPC for its robustness against unknown model uncertainties and external disturbances resulting from inter- and intra-subject variability, and uncertainty of the parameters in Table I due to varying disease stages and exercise levels. We change the two most sensitive parameters, d_{fes} and Grs, to simulate the most challenging uncertainty situations and evaluate robustness of MMPC performance. The baseline output in region two is used as the set point. For a +/−5%, +/−10%, +/−20% change of dfₑₛ from its nominal value from cycle 10 to 160, the disturbance rejection results are shown in FIG. 6, and the open-loop results using the constant baseline control inputs for changing +/−20% from the nominal parameter values are shown by dotted line for reference. The MMPC offset compared with open-loop performance, but there is a large offset of HR with a 20% change of dfₑₛ. This is because more weight is assigned to MAP than HR in the MPC objective function. Another reason is that the models of HR. Similarly, the disturbance rejection results by changing GTs are shown in FIG. 7. A small change of GTs leads to a large variation in HR. The MMPC converges to the set point with very small offset in 5% change of GTs, but a large oscillation was observed for HR when the GTs was changed by +/−20% with current setting of tuning weight. In this case, HR is more sensitive to external stimulus, requiring additional models for each operating region for HR dynamics. The results in FIG. 8 show how the MMPC responds to change in the two sensitive parameters simultaneously. The output results using the constant baseline control inputs is presented for comparison. 10% change of both parameters are induced from cycle 10 to 160 and the system is brought back to its nominal state from cycle 160 to 300. The MMPC can adjust inputs to correct for these disturbances by automatically picking the correct prediction models.

### C. Event Adaptation and HIL Demonstration

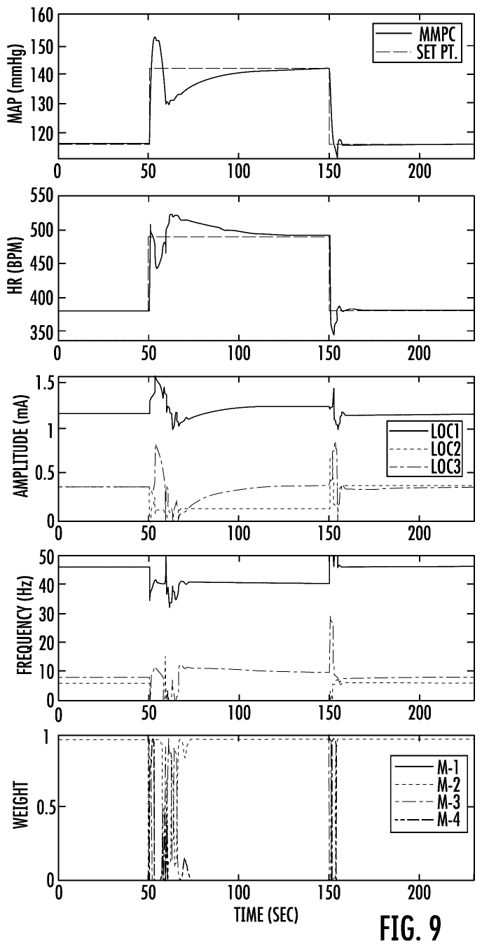

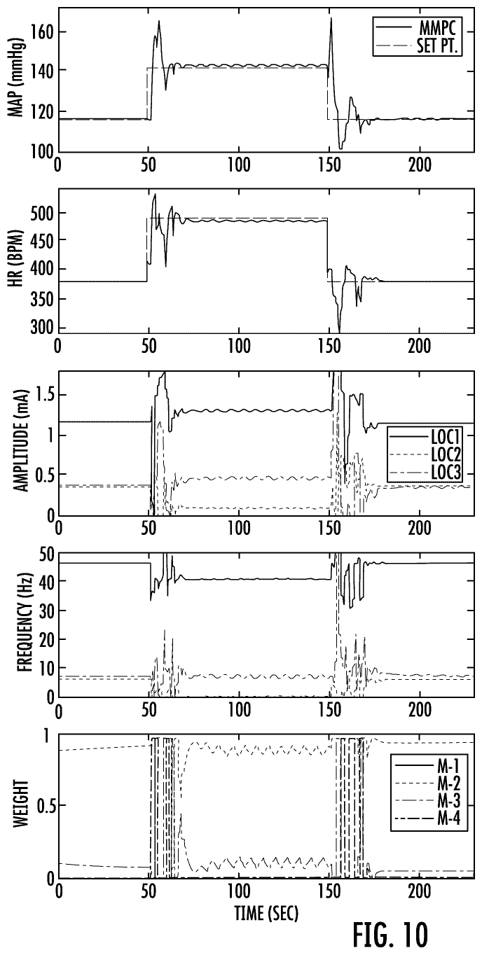

The resting HR and MAP increase within a few seconds when an acute exercise begins. Keeping the same setpoint for each output may result in unsatisfactory control decisions and cause severe side effect for patients. Therefore, our closed-loop ‘rest’ and ‘exercise’ modes with different output setpoints. In this test, the ‘rest’ and the ‘exercise’ setpoints are the HR and MAP obtained by simulating the full system model using the nominal and nominal-exercise parameter set described in Table I, respectively. To simulate the slow dynamic transition from rest to exercise state, each change of Rₛₗ₂ and dfₑₛ is constant. The main purpose of HIL simulation is to demonstrate how our control system can be successfully implemented as provide a pre-clinical assessment of the algorithm's safety and primary obstacle to overcome when implementing the MMPC in a real system is the computation time, which is directly related to the size of the optimization problem in the controller. The larger the order of the linear models and the larger the prediction and control horizon are, the longer the computation time will be. Ideally, the computation time should be less than the smallest period of a cardiac cycle. However, by testing the CVXOPT solver, we found the maximum computation time for our controller with prediction horizon 20 and control horizon 10 is around 0.5 seconds. This is much longer than a cardiac period of a normal rat, which is around 0.15 seconds. Thus we modified the MMPC to make control decisions every five cardiac cycles. During the time period for the controller to finish the optimization problem, the Raspberry Pi keeps receiving output measurements, setpoints, and a trigger signal indicating the end of each cardiac cycle with a sample time of 0.01 second. Before implementing MMPC algorithm on Raspberry Pi, we first conduct a closed loop simulation in MATLAB to test its feasibility in sampling at five cardiac cycles. FIG. 9 depicts an example of event adaptation performance of MMPC in MATLAB. An acute exercise starts at 50 seconds and ends at 150 seconds. The MMPC can track the setpoint for ‘exercise’ mode, as well as the ‘rest’ setpoint after the exercise. In this state and the exercise-induced dynamic changes are treated as unmeasured disturbance. The MMPC performance demonstrates that our control to accommodate latency in HIL implementation. Then, the same scenario of event adaption for MMPC using MATLAB is tested for HIL implementation, which is shown in FIG. 10. The small mismatch between the simulation results in MATLAB (FIG. 9) and HIL/SIMULINK (FIG. 10) is due to the following:
equations in MATLAB and Simulink (for HIL); (b) in HIL mode, packet drops in the TCP/IP communication between the Raspberry Pi and the SIMULINK model on the desktop computer; (c) in HIL mode, output measurements, setpoints, and a trigger signal received by the Raspberry PI indicating the end of each cardiac cycle with a sample time of 0.01 second, during the time that the controller is solving the optimization problem.

In this aforementioned formulation, we have proposed a MMPC algorithm for the control of HR and MAP of a rat cardiovascular model using VNS. Compared to traditional MPC with a single local model, the MMPC can capture the nonlinear characteristics of complex system and has the adaptive ability derived from the recursive Bayesian weighting theory. The model bank generates piece-wise linear models based on the range of MAP, which enables the MMPC to control the MAP of a hypertensive rat model the augmented state space models enhances the robustness of MMPC to unmeasured disturbance due to inter and intrasubject variability. We have demonstrated and evaluated the performance of MMPC for regulating HR and MAP using setpoint tracking, disturbance rejection and event adaptation.

Compared with the nonlinear MPC previously developed in our work, it is easier to implement MMPC as an embedded controller due to the well-developed algorithm for linear system and lower computational expense. The feasibility of the MMPC to be implemented as an embedded controller has been for future implementation of our MMPC for pre-clinical and clinical studies. Establishing closed-loop stability of the MMPC algorithm simulations. The proposed MMPC incorporates a discrete time, constrained quadratic optimization objective function, and a weighted model approximation of a nonlinear system following a switching logic. A stability analysis would be composed of two objectives: demonstrate a stability criterion for MMPC with model switch and show the convergence has been investigated in [36]. Stability of MMPC algorithms are especially challenging at the point when a model switch occurs. Multiple Lyapunov functions [37] have found wide utility in stability of hybrid systems, and this method is further extended in [38] for MMPC. Another stability concern is to show that the error between the weighted model and the boundary tolerance, a better baseline value used in the integral term of the MPC objective function, or more models that span the HR dynamics may reduce oscillation and improve the performance of MMPC.
[1] Li, Meihua, et al., “Vagal nerve stimulation markedly improves long-term survival after chronic heart failure in rats,” Circulation, vol. 109, no. 1, pp. 120-124, 2004.
[2] Carpenter, Alexander, et al., should we treat it?” International journal of cardiology, vol. 201, pp. 415-421, 2015.
[3] Plachta, Dennis T T, et al., “Blood pressure control with selective vagal nerve stimulation and minimal side effects,” Journal of neural engineering, vol. 11, no. 3, p. 036011, 2014.
[4] Yuan, Hsiangkuo, et al., “Vagus nerve and vagus nerve stimulation, a comprehensive review: part II,” Headache: The Journal of Head and Face Pain, vol. 56, no. 2, pp. 259-266, 2016.
[5] Premchand, Rajendra K, et al., “Autonomic regulation therapy via left or right cervical vagus nerve stimulation in patients with chronic heart failure: results of the ANTHEM-HF trial,” Journal of cardiac failure, vol. 20, no. 11, pp. 808-816, 2014.
[6] Zannad, Faiez, et al., “Chronic vagal stimulation for the treatment of low ejection fraction heart failure: results of the Neural Cardiac Therapy for Heart Failure (NECTAR-HF) randomized controlled trial,” European heart journal, vol. 36, no. 7, pp. 425-433, 2015.
[7]+old, Michael R, et al., “Vagus nerve stimulation for the treatment of heart failure: the INOVATE-HF trial,” Journal of the American College of Cardiology, vol. 68, no. 2, pp. 149-158, 2016.
[8] Waninger, Matthew S, et al., “Electrophysiological control of ventricular Pacing and Clinical Electrophysiology, vol. 23, no. 8, pp. 1239-1244, 2000.
[9] Zhang, Youhua, et al., “Optimal ventricular rate slowing during atrial American Journal of Physiology-Heart and Circulatory Physiology, vol. 282, no. 3, pp. H1102-H1110, 2002.
[10] Tosato, Marco, et al., “Closed-loop control of the heart rate by electrical stimulation of the vagus nerve,” Medical and Biological Engineering and Computing, vol. 44, no. 3, pp. 161-169, 2006.
[11]+reenwald, Elliot, et al., “A bidirectional neural interface IC with chopper stabilized BioADC array and charge balanced stimulator,” IEEE transactions on biomedical circuits and systems, vol. 10, no. 5, pp. 990-1002, 2016.
[12] Ugalde, Hector M Romero, et al., “Model-based design and experimental validation of control modules for neuromodulation devices,” IEEE Transactions on Biomedical Engineering, vol. 63, no. 7, pp. 1551-1558, 2015.
[13] Romero-Ugalde, Hector M, et al., “A novel controller based on state-transition models for closed-loop vagus nerve stimulation: Application to heart rate regulation,” PloS one, vol. 12, no. 10, p. eo186068, 2017.
[14] Yao, Yuyu and Kothare, Mayuresh V, “Model Predictive Control of Selective Vagal Nerve Stimulation for Regulating Cardiovascular System,” 2020 American Control Conference (ACC), pp. 563-568, IEEE.
[15] Noble, Dennis, The Journal of Physiology, vol. 160, no. 2, pp. 317-352, 1962
[16] Park, James H, et al., “Investigating the effects of brainstem neuronal adaptation on cardiovascular homeostasis,” Frontiers in neuroscience, vol. 14, p. 470, 2020.
[17] Tao, Xiangyuan, et al., “Multiple model predictive control for large Information Sciences, vol. 328, pp. 115-126, 2016.
[18] Urich, Matthew, et al., “Piecewise linear model predictive control of a rapid pressure swing adsorption system,” AIChE Journal, vol. 66, no. 11, p. e16998, 2020.
[19] Athans, et al., “The stochastic control of the F-8C aircraft using a multiple model adaptive control method,” IEEE Trans. Auto Cont., 22, 768-780 (1977).
[20] Rao, Ramesh R, et al., “Experimental studies on multiple-model predictive control for automated regulation of hemodynamic variables,” IEEE Transactions on biomedical engineering, vol. 50, no. 3, pp. 277-288, 2003.
[21] Yao, Yuyu and Kothare, Mayuresh V, “Model-based closed-loop design of neuromodulation technology for heart rate and blood pressure control,” submitted to IEEE Transactions on biomedical engineering.
[22] Ursino, Mauro, “Interaction between carotid baroregulation and the pul-sating heart: a mathematical model,” American Journal of Physiology-Heart and Circulatory Physiology, vol. 275, no. 5, pp. H1733-H1747, 1998.
[23] Mahdi, Adam, et al., PooS computational biology, vol. 9, no. 12, p. e1003384, 2013.
[24] Magosso, Elisa and Ursino, Mauro, “Cardiovascular response to dynamic aerobic exercise: A methematical model,” Medical and Biological Engineering and Computing, vol. 40, no. 6, pp. 660-674, 2002.
[25] Sadashivaiah, Vijay, et al., “Modeling the interactions between stimulation and Journal of Computational Neuroscience, vol. 45, no. 3, pp. 193-206, 2018.
[26] Capilupi, Michael J, et al., “Vagus nerve stimulation and the cardiovascular system,” Cold Spring Harbor oaboratory Press, vol. 10, no. 2, p.a034173, 2020.
[27] Mayet, Jamil and Hughes, Alun, “Cardiac and vascular pathophysiology in hypertension,” Heart, vol. 89, no. 9, pp. 1104-1109, 2003.
[28] Cingolani, Oscar H, et al., “Increased systolic performance with diastolic dysfunction in adult spontaneously hypertensive rats,” vol. 41, no. 2, pp. 249-254, 2003.
[29] Nobrega, Antonio C L, et al., “Neural regulation of cardiovascular response to exercise: role of central command and peripheral afferents,” BioMed research international, vol. 2014, 2014.
[30] Seals, Douglas R., Journal of Applied Physiology, vol. 67, no. 5, pp. 1801-1806, 1989.
[31] Yeung, Pollen K F, et al., “Effect of acute exercise on cardiovascular hemodynamic and red blood cell concentrations of purine nucleotides in hypertensive compared with normotensives rats,” Therapeutic advances in cardiovascular disease, vol. 7, no. 2, pp. 63-74, 2013.
[32] Chandler, Margaret P and Dicarlo, Stephen E, “Acute exercise and gender alter cardiac autonomic tonus differently in hypertensive and normotensive rats,” American Journal of Physiology-Regulatory, Integrative and Comparative Physiology, vol. 274, no. 2, pp. R510-R516, 1998.
[33] Pianost, Francesca, Environmental Modeling and Software, vol. 67, pp. 1-11, 2015.
[34] P. Van Overschee and B. De Moor, “N4sid: Subspace algorithms” Automatica, vol. 30, no. 1, pp. 75-93, 1994.
[35] Andersen, Martin, et al., “Interior-point methods for large-scale cone programming,” Optimization for machine learning, vol. 5583, 2011.
[36] Costa, Eduardo F, and Joao B R do Val, “Costa, Eduardo F., and Joao for nonlinear systems and stability of the approximating controls,” IEEE transactions on automatic control, vol. 54, no. 4, pp. 881-886, 2009.

### Example 2—In Vivo Device and Model Simulations and Measurements

Data for this study were obtained from five male Wistar rats (350-400 g bodyweight). The rats were anesthetized initially with 2-4% vol. isoflurane. Carprofen (5 mg kg-1 body weight) was administered subcutaneously for analgesia. Anesthesia was maintained with 1-2% vol. isoflurane, regulated by the respiration rate. The rats were placed on a regulated, electrically isolated heat mat and received subcutaneous saline (3 ml/h).

In the rat, the aortic depressor nerve (ADN) follows alongside the VN within the vagal nerve bundle. The BP signal is frequency encoded in a phasic form and travels as a slow volley of activity along the 400 unmyelinated fibers of the ADN. Selective electrical stimulation of these nerve fibers, when isolated from the VN, has been shown to activate the baroreflex and reduce the BP in rats.

The left neurovascular bundle of the vagal nerve and the common carotid artery were exposed through a ventral neck incision. The carotid artery was ligated distally and temporarily interrupted proximally with an aneurysm-clip. A tip catheter was inserted and fixed in the Waynforth position in the aorta and the aneurysm clip was removed. A multichannel cuff electrode (MCE) was wrapped around the vagal nerve bundle without any pre-alignments of electrodes and nerve. Three ECG needle electrodes were inserted subcutaneously in the left and right forepaw and in the tail (ground). After an initial recording to locate the electrodes closest to the baroreceptive fibers (baroreceptive electrodes), stimulation parameters were tested in arbitrary order to avoid adaptation processes. The stimulus parameters were tested primarily on baroreceptive electrodes and, for control purposes, through electrodes that were not located near the barofibers (non-baroreceptive electrodes).

The MCE and ECG needle electrodes were connected to a PZ3 system (Tucker Davis Technology, FL, USA), which contains low noise pre-amplifiers (noise floor 0.9 pVRMS) for the signal conditioning, attached to an RZ2-module, which holds two digital signal processors (DSPs) and allows digital/analogue inputs/outputs to preprocess the signals. The RZ2 was connected to a PC. The PZ3 pre-amplifier was set to monopolar recording of each of the 24 working electrodes and the two reference electrodes at a sample rate of 12 kHz. A notch filter (50 Hz) was applied to the data. Except for the notch filter, the data were stored unfiltered. However, we applied a band-pass filter (Butterworth fourth order, 20 to 300 Hz) for the signals fed into the real-time analysis. The sample rate and the filter settings were the same as for the electrodes. Signal processing on the PC included real-time calculation of true-tripoles of the electrodes, filtering and coherent averaging to detect the baroreceptive activity. The coherent averaging was triggered by the rising edge of the ECG signal with a threshold value two times greater than noise level.

Current controlled, charge balanced stimulation with biphasic rectangular pulses, was generated and modified in the RZ2 module and fed into a voltage-to-current-converter at a D/A conversion rate of 24 kHz and 16-bit resolution. The center electrode of the baroreceptive tripole was used as the cathode against the two large peripheral ring electrodes as anodes. We first located the tripole that showed baroreceptive activity after filtering and coherent averaging. We then selected the center electrode of this recording tripole and applied each combination of stimulation parameters five times in an arbitrary order.

FIG. 5 shows the closed-loop performance of our designed controller. For the first 150 cardiac cycles, the set point r (k) was set to 356 (bpm) and 150 (mmHg) for HR and MAP, respectively. For the next 150 cardiac cycles, the set point was set to 393 (bpm) and 129 (mmHg) for HR and MAP, respectively. Finally, for the final 150 cardiac cycles, the set point was set to 377 (bpm) and 143 (mmHg) for HR and MAP, respectively. For the next 150 cardiac cycles, the set point was set to 393 (bpm) and 129 (mmHg) for HR and MAP, respectively. Finally, for the final 150 cardiac cycles, the set point was set to 377 (bpm) and 143 (mmHg) for HR and MAP, respectively. For the controller we set Nₚ=20 and Nc=10 for this simulation. For each setpoint, we ran the simulation for 100 cardiac cycles longer (150 cardiac cycles as opposed to 50 cardiac cycles in previous closed-loop simulations) to ensure the convergence of the offset-free closed-loop control formulation. As shown in FIG. 7, the controller is able to achieve offset-free control within the first 50 cardiac cycles of each set point change. As the controller approaches each set point, there is an overshoot of each target for the first 20 cardiac cycles of the set point change. Notably, the optimized VNS parameters are generally smooth and constant near the steady state.

The most common control approach reported in the literature is based on proportional integral (PI) control. While the traditional PI control algorithm is easy to implement, it does not take constraints into consideration, i.e. the bounds on the pulse amplitude, width, frequency and the tachycardiac and bradycardiac states. In addition, it is non-trivial to design a PI controller for a multiple-input-multiple-output system which has substantial interactions between inputs and outputs. Model predictive control (MPC) has already been used as a way to overcome these difficulties and has been widely tested on a variety of biomedical systems, such as the control of mean arterial pressure under anesthesia [14], control of blood glucose concentration for people with type 1 diabetes [15], and control of movement after spinal cord injury [16]. This control algorithm is most appropriate for the development of a closed-loop VNS system, since it can simultaneously manipulate multiple constraints and controlled variables within a single optimal control law and is able to deal with high non-linearity caused by threshold and saturation effects involved in neurotransmitter-receptor dynamics.

In this study, we develop a model-based framework for a multi-location closed-loop VNS system. The proposed frame-work includes 1) an in silico model, which is used to sim-ulate cardiovascular responses of normal and diseased rats, 2) a nonlinear model predictive control (NMPC) algorithm, which controls the HR and MAP synchronously with each cardiac beat by independently manipulating pulse amplitude and frequency in three different neurostimulation locations. The in silico model is derived by compartmentalizing the various physiological components involved in the closed-loop cardiovascular system (CVS) of rats with intrinsic baroreflex regulation, which accounts for the discrete events associated with opening and closing of the heart valves during each cardiac cycle. We consider this in silico model to represent the “true” CVS. A reduced order computationally efficient model is derived from the “true” in silico system dynamics by averaging the intra-beat dynamics of each hemodynamic variable and by using a simplified MAP-based baroreflex model. This reduced order model is used as the “predictor” in the NMPC algorithm. The parameters of the reduced model are determined by a data-driven approach. Our results show the feasibility and usefulness of the proposed closed-loop design for the regulation of HR and MAP.

### Example 3—Simulation Model

A mathematical model to predict rat cardiovascular response to VNS is needed for designing and validating the efficacy and safety of the closed-loop VNS device. Similar to our previous study [17], we build such a system-level model by either combining models describing part of the overall system or replacing part of a published model by a more detailed compartment model published by other authors. Several modifications of the earlier model are introduced: 1) division of the systemic circulation into the parallel arrangement of two distinct segments, representing the circulations in the upper and lower body; 2) inclusion of the conductance map in the device model to capture the interactions between stimulation and physiologically induced action potentials in different nerve fibers; 3) modification of adjustable stimulation parameters from pulse width, pulse frequency to pulse amplitude, pulse frequency since the amplitude is a stronger predictor of the cardiovascular response to VNS than pulse width, as well as the availability of data on these responses; 4) inclusion of the total stressed blood volume as the other effector responding to the sympathetic drive. The modified model is summarized in the following subsections.

TABLE III
Abbreviations of subscripts in CVS model

| Abbreviation | Name |
|---|---|
| au | Arteries in upper body |
| al | Arteries in lower body |
| vu | Veins in upper body |
| vl | Veins in lower body |
| av | Aortic valve |
| mv | Mitral valve |
| su | Peripheral resistance in upper body |
| sl | Peripheral resistance in lower body |
| sa | Arterial resistance |
| sv | Venous resistance |
| lh | Left heart |

### Cardiovascular Model

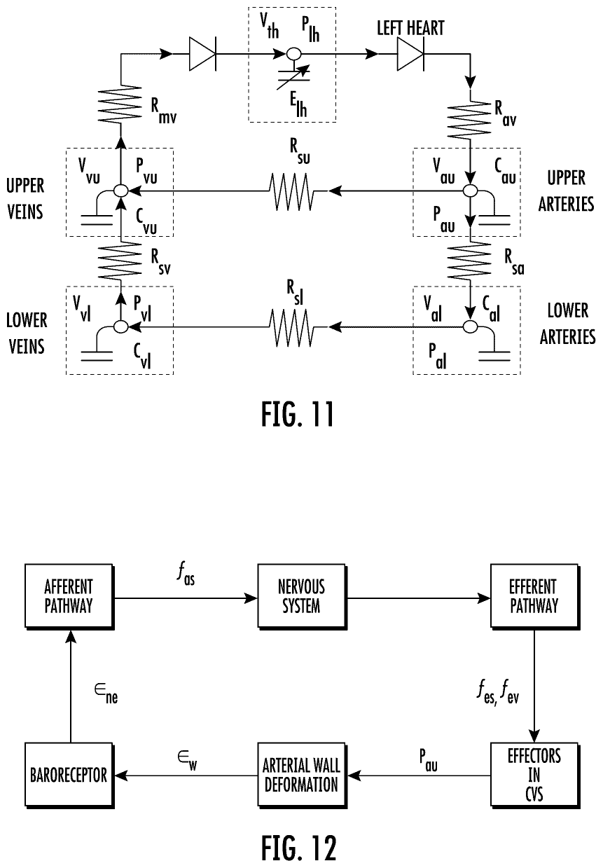

The rat cardiovascular system (CVS) model, adapted from [18], includes five compartments representing the left heart, the arteries and the veins in the upper and lower body. The right heart and the pulmonary circulation is modeled by a capacitance, which is added to the venous capacitance in the upper body. The model mimics an electrical RC-circuit (see FIG. 11). The blood pressure in the four arterial and venous compartments, and the total volume of the left heart follow Kirchhoffs law where the abbreviations of the subscripts are listed in Table III. R is resistance; P is pressure; V is volume; C is constant compartment compliance. Pumping is achieved by a time-varying elastance of the left heart, which transitions between an exponential function representing diastole and a linear function representing the end of the systole [19]:

$${P}_{lh} = φ ( t ) {E}_{max , lh} ( {V}_{lh} - {V}_{u , lh} ) + [ 1 - φ ( t ) ] Po , lh ( ekE , lhVlh - 1 )$$

$$0 \leq φ ( t ) \leq 1$$

where E_{max,lh} is the maximal contraction elastance of the left heart; V_{u,lh} is the unstressed volume of the left heart; P_{o,lh} and k_{E,lh} are constant parameters for the exponential function. The term p(t) represents the periodic “activation function”. When φ(t)=1, the left heart is at maximum contraction, when p(t)=0, the left heart is at complete relaxation. Finally, the heart valves are modeled as diodes, which results in four different time intervals in a normal cardiac cycle.

### B. Baroreflex Model

The baroreflex model is adapted from [19]-[21]. It consists of the baroreceptor, afferent pathway, efferent pathway, and the effectors in CVS (see FIG. 12).

The baroreceptors are stretch-sensitive fibers located in the carotid sinus which convert the arterial pressure to afferent firing rates. A model with two Voigt bodies and a spring in series is used to describe the stretching dynamics of the baroreceptive nerve endings:

$$\frac{d {ϵ}_{1}}{dt} = - ( {α}_{1} + {α}_{2} + {β}_{1} ) {ϵ}_{1} + ( {β}_{1} - {β}_{2} ) {ϵ}_{2} + ( {α}_{1} - {α}_{2} ) {ϵ}_{w} \qquad \text{?}$$

$$\frac{d {ϵ}_{2}}{dt} = - {α}_{2} {ϵ}_{1} - {β}_{2} {ϵ}_{2} + {α}_{2} {ϵ}_{w} \qquad \text{?}$$

$${ϵ}_{ne} = {ϵ}_{w} - {ϵ}_{1} \qquad \text{?}$$

$$\text{?} \text{indicates text missing or illegible when filed}$$

Where E₁ and E₂ are the relative displacements within each Voigt body; a₁, a₂, β₁, and β₂ are nerve ending constants; Ene denotes nerve ending strain; E_{w} refers to the strain of the arterial wall which is described by a static sigmoid function of arterial pressure. The firing rate in the afferent pathway is calculated by a leaky integrate-and-fire model represented by

$${f}_{as} = \{ \begin{matrix}{[ \frac{{C}_{m}}{{g}_{leak}} ln ( \frac{{I}_{ne}}{{I}_{ne} - {g}_{leak} {V}_{th}} ) + {t}_{ref} ]}^{- 1} & if {I}_{ne} > {g}_{leak} {V}_{th} \\ 0 & otherwise\end{matrix} . \qquad \text{?}$$

$$\text{?} \text{indicates text missing or illegible when filed}$$

where Iₙₑ is the current stimulus injected into afferent fibers, modeled as a linear function of Ene; gleak is a leakage conductance; Cm denotes the membrane capacitance; Vth is a
given voltage threshold, and tref is a refractory period.

The afferent firing rate is translated into (1) an efferent sympathetic firing rate using a monotonically decreasing function; and (2) an efferent vagal firing rate using a monotonically increasing function with an upper saturation, respectively. These efferent signals are delivered into the end organs in the CVS. The regulation effects include changes in peripheral resistance in upper and lower body, heart contractility, total stressed blood volume and heart period. The response of the heart period includes a balance between the sympathetic and vagal activities. The heart period changed by sympathetic stimulation (Tₑₛ) includes a time delay, a logarithmic static function and a low-pass first-order dynamic, while the response of heart period to vagal activities (Tₑᵥ) shows a positive linear relationship. Finally, the heart period is achieved by linear interaction between sympathetic and vagal responses. The dynamics for changes in heart period are given in (11)-(13).
Dynamics of peripheral resistance, heart contractility, and total stressed blood volume ar similar to those of Tₑₛ.

$$\frac{d {T}_{e s}}{d t} = \qquad − \frac{{T}_{e s} − {T}_{o}}{{τ}_{T e s}} + \frac{{G}_{Tes} ln ( max \{ {f}_{e s} ( t − {D}_{Tes} ) − {f}_{es , min} , 1 \} )}{τ {T}_{e s}}$$

$$\frac{{dT}_{ev}}{dt} = \frac{− ( {T}_{e v} − {T}_{o} ) + {G}_{Tev} {f}_{e v} ( t − {D}_{Tev} )}{{τ}_{Tev}}$$

$$T = {T}_{e s} + {T}_{ev} - {T}_{o}$$

corresponding time constant, gain and delay in sympathetic (vagal) pathway. Tₒ is intrinsic heart period.

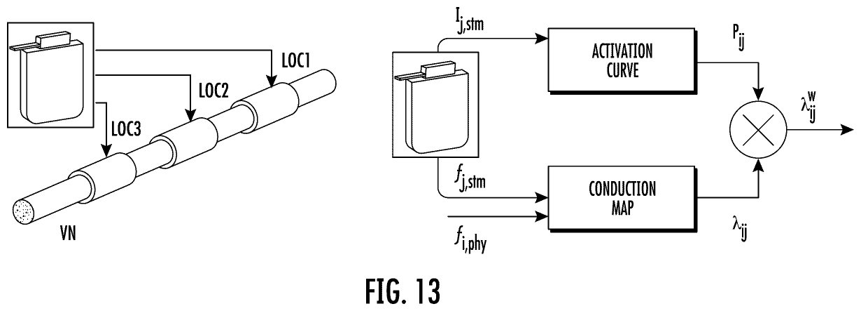

A device model is developed to predict the response of firing rate of different fibers to VNS pulse trains. In the rat, the barofibers are bundled into the aortic depressor nerve, which follows alongside the vagal nerve within the vagal nerve bundle [4]. The cervical sympathetic nerve runs separately from the vagus in rats. However, other mammals, including dogs, pigs and humans have a cervical vagosympathetic trunk [22], [23] and detailed microdissection to identify and isolate the sympathetic trunk, the aortic depressor nerve, and the vagal nerve may not be safe or practical during human VNS surgery [24]. There is also a big difference in cardiovascular control between the left and right VNS. In rats, left-sided cervical VNS caused stronger bradycardiac, hypotensive, and tachypneic effects than right-sided VNS [25]. However, this contrasts with findings in dogs [26] that demonstrated greater bradycardia with right-sided than left-sided VNS synthetic fibers. Each type of fiber distributes nonhomogenously in different stimulation locations (FIG. 13, Left) and the total firing rate of each type of fiber is calculated as the weighted sum of its firing rate in each stimulation location:

$${f}_{i} = {\sum }_{j = 1}^{n} {L}_{j} {C}_{ij} {f}_{ij} + ( 1 − {\sum }_{j = 1}^{n} {C}_{ij} ) {f}_{i , phy} \qquad [ EQUATION 14 ]$$

where fi is total firing rate of fiber type i; n is number of stimulation locations; Lj represents the on-off condition of locationj; Cᵢⱼ is concentration of fiber type i in location j; fij is firing rate of fiber type i in location j; fi,_{phy} is physiological firing rate of fiber type i unaffected by external stimulation. The proposed device model for activating each fiber type in each stimulation location (FIG. 13, Right) consists of a nerve fiber activation curve and a conduction map. The model assumes that the width of the stimulation pulse train is constant, and that the pulse amplitude and frequency varies. The activation curve represents the proportion of nerve fibers recruited by stimulation of a particular amplitude using a static sigmoid function.

$${P}_{ij} ( {I}_{j , s t m} ) = \frac{{e}^{\frac{{I}_{j , s t m} − {I}_{ij , mid}}{{k}_{{I}_{ij}}}}}{1 + {e}^{\frac{{I}_{j , s t m} − {I}_{ij , mid}}{{k}_{{I}_{ij}}}}} \qquad [ EQUATION 15 ]$$

where Pij refers to activation probability of fiber i in stim-ulation location j; Ij,stm is the amplitude of VNS pulse train; I_{ij,mid} is the value of amplitude at central point of the sigmoid function, and k_{I}ij is the slope at the central point. A conduction reliability (λᵢⱼ) represents the percentage of total action potentials conducted to the somatic end of the nerve fiber by considering the interactions between stimulation and physiologically induced action potentials. As the frequency of the stimulus and/or rate of the physiological input increases, conduction reliability decreases due to collisions between these two types of action potentials, as well as the inter- and intra-signal loss of excitability due to neural refractory period. We adopted the conduction map from [28] by fitting the data from the mechanistic model to a linear combination of physiological frequency and external stimulation frequency. The relationship between stimulation parameters and fiber firing rate at each stimulation location can be represented by the following equation:

$${f}_{ij} = ( {f}_{i , p h y} + {f}_{j , s t m} ) {λ}_{ij}^{w} + {f}_{i , p h y} ( 1 − {P}_{ij} ) \qquad ( 16 )$$

wheref_{j,stm} refers to firing rate of the external stimulus at location j; λ^{w}=Pᵢⱼλᵢⱼ refers to weighted conduction reliability.

### D. Model Parameter Estimation

Since the model was based upon several compartment models previously developed in [18]-[20], we either used the same parameter values as those used in the original models or adapted some values to produce simulations that best matched experimental data associated with nominal, diseased, and exercise states of rats. We describe below the various steps used in this model validation. The results of the model validation and matching with experimental data are provided in section IV A.
To simulate the nominal states, the parameters in the CVS model were adapted from those in [18] to match the experimental data of young healthy rats in [29]. The parameters in the baroreceptor model are the same as those used in [20]. The parameters in the efferent pathway are the same as those used in [19], while the parameters in the transfer function related to effector dynamics are modified by multiplying the original value by a scaling constant which indicates the difference in heart period and blood volume between a rat and a hu-man. Three stimulation locations are identified for the device model. The parameters representing the fiber concentration, the activation curve and the conduction map in baroreceptive and non-baroreceptive locations are obtained by matching the simulated change of HR and MAP by VNS to experimental data in [4], [30], respectively. Percent of vagal fibers which are not contained in these two locations are set to the third stimulation location. Hypertensive heart disease is used as an example to show the feasibility of closed-loop control because VNS has an acute effect on reducing hypertension. Other cardiovascular diseases responsive to VNS require activation of long-term mecha-nisms which are not captured by the model. Drug-resistant hypertension is related to increased arterial stiffness, vascular remodeling, and increased sympathetic activity and decreased vagal activity. Most longstanding hypertension ultimately leads to heart failure and diastolic dysfunction is characterized as an early marker of heart damage in hypertension. To simulate the diseased state, we introduce an offset in sympathetic and vagal activity, increase the cross-section area of the artery in relaxation state and modify the gain of each effectors to match the data in [31].

Acute exercise triggers multiple physiological responses, including redistribution of blood flow and modification of sympathetic and vagal activities by central command. To address these issues, we separate the peripheral resistance in the lower body into two parallel resistors: Rsl1 representing the resting muscle resistance and Rsl2 representing the active muscle resistance. The exercise states are determined by a combination of an offset to sympathetic activity and forced change in active muscle resistance (Rsl2). The experimental hemodynamic data of Sprague-Dawley rat during last minute of treadmill exercise from [32] is used as reference for tuning the parameters for modeling the nominal rat. When estimating parameters for an exercised hypertensive rat, we also selected different values for gain parameters of the effectors to match the arterial pressure and HR experimental data for spontaneous hypertensive rat (SHR) during the last minute of the treadmill exercise in [33].

### III. CONTROLLER DESIGN

The control objective is to regulate simultaneously MAP and HR by the automated modification of neural activities involved in the vagal bundles. There are loop interactions between these control variables. Changes made to MAP will change the baroreceptive activities, and hence the HR. Similarly, changes made to HR will change the cardiac output, and hence influence the MAP. It is difficult to decouple these loops into multiple SISO models. The multivariable control algorithm chosen, namely, NMPC, is able to handle and opti-mize these interactions in closed loop. The MIMO model itself characterizes the nonlinearities of the physiological system as a constraint to predict the coupled multivariable change of outputs and calculate the optimal inputs.

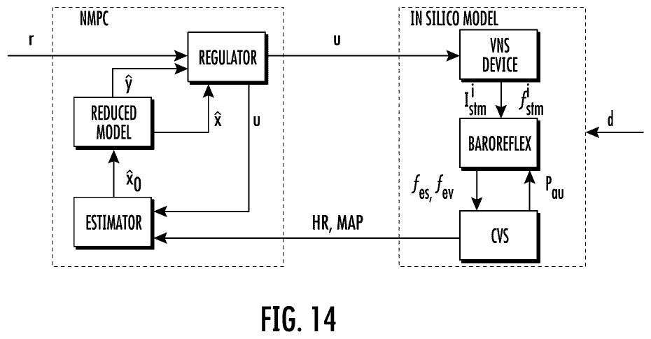

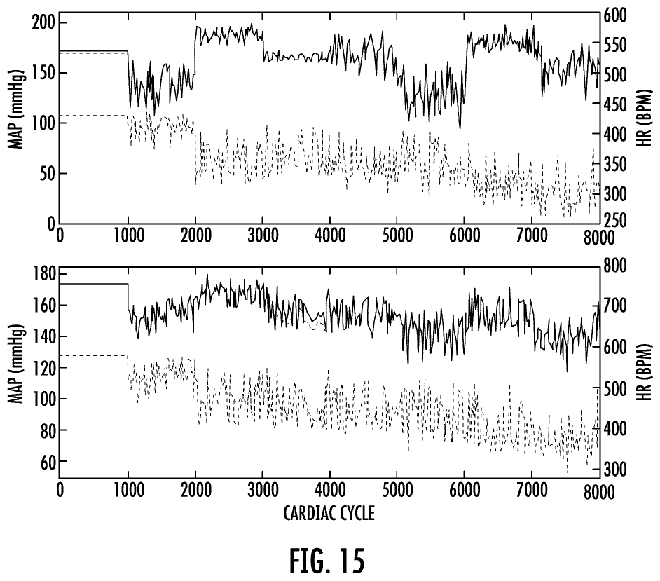

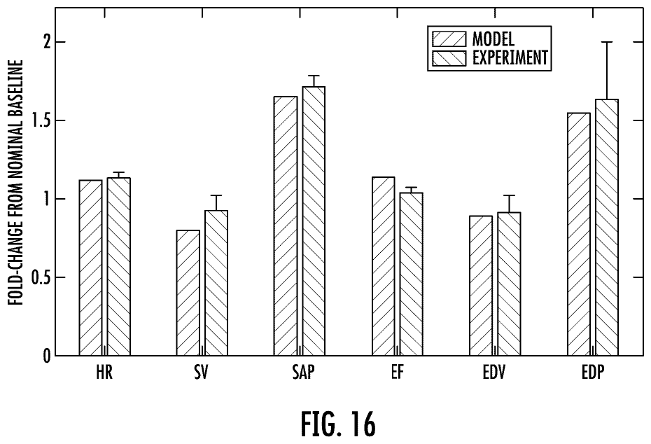

However, the in silico simulation model previously described is time-varying and not differentiable, which makes it computationally expensive and practically infeasible to implement in NMPC. A reduced time-invariant differentiable model was derived to capture the behavior of the CVS by using representative mean values that are invariant within the cardiac cycle. The NMPC uses the reduced model to predict the future outputs of the system to be controlled but is tested by the full simulation in silico model for setpoint tracking and disturbance rejection (see block diagram in FIG. 14).

### A. Reduced Model

The reduced model has the same components as the previously described full simulation model. The primary difference is that the reduced model only considers the inter-beat dynam-ics and ignores the intra-beat dynamics. Some simplifications of the cardiovascular and baroreflex model are made, while the device model is kept the same as the full simulation model
The CVS model is reduced to a three-compartment model, representing the artery, the vein, and the left heart. The time-varying elastance of the pumping heart is replaced by the cardiac output as a function of heart period, venous and arterial pressure using the integration method developed in [18]. The reduced cardiovascular model can be finally represented by the following differential equations:

$$\frac{d {P}_{v c}}{d t} = \frac{{P}_{a o} − {P}_{v c}}{{C}_{v c} {R}_{s y s}} − \frac{Q}{{C}_{v c}}$$

$$\frac{d {P}_{a o}}{d t} = \frac{{P}_{v c} − {P}_{a o}}{{C}_{a o} {R}_{s y s}} + \frac{Q}{{C}_{a o}}$$

$$Q = \frac{1}{T} ( \frac{{P}_{v c}}{{E}_{f}} − \frac{{P}_{a o}}{{E}_{e}} )$$

where P_{vc}, Pₐₒ are the pressure of the veins and the arteries,
respectively; C_{vc}, Cₐₒ are the compliance of the veins and the arteries, respectively; R_{sys} is the systemic resistance in the peripheral circulation; Q is the cardiac output; Ef and Ee are the averaged elastance of the left heart during filling and ejection, respectively. The previously described baroreflex model is sensitive to the functional form of arterial pressure and includes a time delay. But the arterial pressure predicted by the reduced cardiovascular model is constant during each cardiac cycle. As a result, a reduced baroreflex model is adapted from [34] to regulate the averaged arterial pressure. The physiological firing rate of the barofibers (fas,phy) is defined as the product of the ratio of the arterial pressure to some predefined setpoint of the arterial pressure (Psp) and a predefined setpoint of the baroreceptive firing rate:

$${f}_{a s , phy} = \frac{{P}_{a o}}{{P}_{sp}} {f}_{a s , sp}$$

The firing rates of sympathetic fibers (f_{es,phy}) and vagal fibers (f_{ev,phy}) are related to the baroreceptive firing rate by the following monotonically decreasing and increasing static curves, respectively.

$${f}_{e s , p h y} = \frac{{f}_{es , max}}{1 + {δ}^{{k}_{e s}}} , {f}_{e v , p h y} = \frac{{f}_{ev , max}}{1 + {δ}^{− {k}_{e v}}} \qquad [ EQUATION 19 ]$$

where f_{es,max}, f_{ev,max} are maximum firing rates of sympathetic and vagal fibers; kₑₛ and kₑᵥ are steepness parameters of sympathetic and vagal pathway; δ=F (f_{as,phy, u})/f_{as,sp} is the normalized baroreceptive activity modified by external stimulation with F (*) representing the device model defined in (14) -(16) and u={Lⱼ, I_{j,stm}, f_{j,stm}}T is the vector of control variables representing the on-off conditions of three stimulation locations and the corresponding amplitude and frequency of the pulse train. Five effectors, representing the heart period, the systemic resistance, the elastance of left heart during filling and ejection, and the total unstressed blood volume are responsive to efferent drive. The change of each effector (θi) is described by the following [35]:

$$\frac{d {θ}_{i}}{d t} = \frac{− {θ}_{i} + {α}_{i} {n}_{e s} + {β}_{i} {n}_{e v} + {γ}_{i}}{{τ}_{{θ}_{i}}} \qquad [ EQUATION 20 ]$$

$$where$$

$${n}_{e s} = \frac{F ( {f}_{e s , p h y} , u )}{{f}_{e s , m a x}}$$

$$and$$

$${n}_{ev} = \frac{F ( {f}_{e v , p h y} , u )}{{f}_{ev , max}} \text{?}$$

$$\text{?} \text{indicates text missing or illegible when filed}$$

are normalized firing activities of sympathetic and vagal fibers with external stimulus, respectively; αi, βi, and γi are the gain parameters; τθi refers to the characteristic time constant. This uniform formulation removes the discontinuity in a logarithmic function and makes the nonlinear optimization tractable.

### B. System Identification

Several of the parameters in the reduced model need to be recalculated to match the dynamics of the full in silico simulation model. The nonlinear grey-box technique was used to identify the reduced model with the MATLAB implementation of the algorithm ‘nl_{greyest}’. The selected parameters to be identified are listed in Table III; the other parameters are kept the same as in the full simulation model. The algorithm estimates the reduced model parameters by minimizing the error between the reduced model output and the output from the full model using the following objective function:

$$\begin{matrix} & {V}_{N} ( η ) = \frac{1}{N} {\sum }_{t = 1}^{N} {ε}^{2} ( t , η ) + \frac{1}{N} λ {η}^{T} R η\end{matrix} \qquad [ EQUATION 21 ]$$

$$s . t . \qquad {η}_{min} \leq η \leq {η}_{max}$$

where t is time, N is the number of data samples, and ε(t, η) is the error between the reduced and full model output; X is a positive constant which trades-off variance versus bias error; R is the weight matrix for variance error; 1 min, 1 max are vectors of the lower and upper bound of each parameter which is selected to be ±5-fold of the corresponding parameter value in the full model. The input-output signals are generated from the full diseased model in both rest and exercise regimes. A uniform distribution is used to generate 50 random values of pulse amplitude and pulse frequency within their range for each of the 8 combinations of stimulation locations. Each perturbation of stimulation configurations lasts for 20 cardiac cycles to capture the dynamic response of the system. The output signals are the cycle-averaged mean arterial pressure (MAP) and heart rate (HR) and the sample time was set to 1 for integration.

The reduced model predictions of the diseased condition in rest and exercise regimes are shown in FIG. 5 as dashed lines, and the data generated by the full diseased model is shown as solid lines. The reduced model has a 85.97% match for the MAP and 92.54% match for the HR for rest regime (and 75.27% and 84.5% for exercise regime). The mismatch between the identified reduced model (“predictor”) and the full in silico model can be expected to be handled by the feedback action of the NMPC with the moving horizon estimator.

### C. Nonlinear Model Predictive Controller

NMPC is based on an optimal control algorithm. At each sample time, it calculates several control actions over a future time horizon by minimizing an appropriate cost function over a future prediction horizon using the reduced model to predict the system response to these control moves. Only the first control action is applied to the system, new measurements of HR and MAP are obtained and the prediction and control horizons are shifted one step forward to compute the next set of optimal control moves.

The objective of NMPC in our VNS context is to bring HR and MAP to its nominal value in both rest and exercise scenarios. Previous studies on closed-loop VNS systems used either the stimulation frequency or amplitude as the only manipulated input, but all stimulation parameters, as well as stimulation locations, affect therapeutic efficacy, and should given constraints on input or state variables. The regulator formulation is as follows:

$$[ EQUATION ( 22 ) ]$$

$$\begin{matrix}{min}_{U} & {\sum }_{k = 0}^{N − 1} ( {‖ {\hat{x}}_{k} − {x}_{s} ‖}_{{Q}_{r}}^{2} + {‖ {u}_{k} − {u}_{s} ‖}_{{R}_{r}}^{2} + {‖ Δ {u}_{k} ‖}_{{R}_{u}}^{2} ) +\end{matrix} {‖ {\hat{x}}_{N} − {x}_{s} ‖}_{{P}_{r}}^{2}$$

$$s . t . \qquad {\hat{x}}_{k + 1} = f ( {\hat{x}}_{k} , {u}_{k} + {B}_{d} {\hat{d}}_{k} , p + {B}_{p d} {\hat{d}}_{k} )$$

$${\hat{y}}_{k} = h ( {\hat{x}}_{k} ) + {C}_{d} {\hat{d}}_{k}$$

$${u}_{min} \leq {u}_{k} \leq {u}_{max}$$

$${F}_{k} {u}_{k} ∈ \{ 0 , 1 \}$$

where k is the current cycle index; N is the prediction horizon; xs and us are steady state value of the states and inputs, which are calculated by a standard target problem for each setpoint of the output; Δuk is the change in input variables uk; Qᵣ, Rᵣ, Rᵤ, and Pᵣ are weighting matrices. The first and second terms in the objective function penalize the deviations of the state predictions from their reference. The third term penalizes large input changes, and the last term provides a terminal constraint to achieve closed-loop stability, where Pᵣ is calculated using the Riccati equation by linearizing the system around the steady-state point. The first two equality constraints represent the reduced model as discussed above, in discrete form. x\hat{ } is the future state predicted using the reduced model; d\hat{ } is the estimated value of the unmeasured disturbance; B_{d}∈R^{Nu×Nd}, B_{pd}∈R^{Np×Nd}, and C_{d}∈R^{Ny×Nd} are input matrices for disturbance. The third constraint imposes a lower bound and upper bound on the inputs to ensure an appropriate stimulation intensity. The last constraint is a binary equality constraint for stimulation locations—each stimulation location is in closed state (value 0) or open state (value 1).

The resulting regulator problem can be treated as a mixed-integer nonlinear program (MINLP). Solving a MINLP is a computationally difficult task since the optimality and speed of convergence cannot be guaranteed. Instead of applying an MINLP solver, we explicitly list the 8 possible combinations of the 3 stimulation locations. For each feasible combination, the explicit values of the 3 binary input variables are inserted into the regulator formulation to give a traditional nonlinear program without binary variables. By repeating the calculation for each feasible combination, the one with the minimal cost is chosen as optimal.

In practical situations, physiological parameters may change with time, which causes unanticipated noise and disturbance on output variables. A moving horizon estimator (MHE) is developed to address these issues using a series of measurements observed over time to estimate the current state and disturbance. The estimation problem is stated as:

$$[ EQUATION ( 23 ) ]$$

$$\begin{matrix}{min}_{X , W , V , D} & ‖ {x}_{{N}_{t}} − {\overline{x}}_{{N}_{t}} {‖}_{{P}_{e}^{− 1}}^{2} + ‖ {d}_{{N}_{t}} − {\overline{d}}_{{N}_{t}} {‖}_{{P}_{{d}_{e}}^{− 1}}^{2} +\end{matrix} {\sum }_{k = 0}^{{N}_{t} − 1} ( {‖ {w}_{k} ‖}_{{Q}_{e}^{− 1}}^{2} + {‖ {υ}_{k} ‖}_{{R}_{e}^{− 1}}^{2} + {‖ Δ {d}_{k} ‖}_{{Q}_{{d}_{e}}^{− 1}}^{2} )$$

$$s . t . \qquad \text{} {\hat{x}}_{k + 1} = f ( {x}_{k} , {u}_{k} + {B}_{d} {d}_{k} , p + {B}_{p d} {d}_{k} ) + {w}_{k}$$

$${\hat{y}}_{k} = h ( {x}_{k} ) + {C}_{d} {d}_{k} + {υ}_{k}$$

$${x}_{min} \leq {x}_{k} \leq {x}_{max}$$

where Nₜ is the length of the moving horizon window; X=[xₒ, xᵢ, . . . , x_{Nt}]T represents the sequence of estimated state variables; W=[wₒ, w₁, . . . , w_{Nt}]T, V=[vₒ, v₁, . . . , v_{Nt}]T, and D=[dₒ, dᵢ, . . . , d_{Nt}]T represent the sequence of estimated state noise, measurement noise, and disturbance, respectively; Pₑ, P_{de}, Qₑ, Qₐₑ, and Rₑ are weighting matrices. The first and second terms in the objective function penalize the arriving cost of state and disturbance variables, respectively. The third and fourth terms minimize the process and measurement noise, respectively. The last term prevents large change in subsequent disturbance estimation. Similar to the regulator problem, the MHE is solved repeatedly at each sampling instance. At each estimation step, the first element from the previous estimation is eliminated and the newest measurement is added into the estimation window. The full simulation model does not intro-duce any process and measurement noise so that the Qₑ and Rₑ are set to a very small value in the optimization problem. By estimating the unmeasured disturbance of model parameters, the steady-state calculation may detect an infeasible problem. In this case, the objective function of the regulator is modified to the following standard MPC formulation:

$${min}_{U} {\sum }_{k = 0}^{N − 1} {‖ {\hat{y}}_{k} − {y}_{s p} ‖}_{Q}^{2} + {‖ Δ {u}_{k} ‖}_{Ru}^{2} \qquad [ ADD EQUATION ( 24 ) ]$$

The computational cost for solving an optimization problem in NMPC depends on its size and formulation. While large horizons are often necessary to capture slow dynamics, the large horizon also significantly increases the computational cost. Thus, a trade-off between optimal solution quality and computational cost is considered in the NMPC design. Here, the control, prediction and the estimation horizons are all set to 10 cycles, which are large enough to allow the controller to provide satisfactory performance.

TABLE IV

| Parameters | Nominal | Hypertens n | VNS |
|---|---|---|---|
| HR (bpm) | 384.22 | 10 | 382.50 |
| | | 428.95 | |
| MAP (mmHg) | 116.04 | 171.53 | 116.20 |
| Ejec. Frac. (%) | 56.87 | 64.33 | 50.15 |
| Stroke Volume (μL) | 144.48 | 114.36 | 123.04 |
| EDV (μL) | 254.05 | 224.6 | 228.06 |

A. Open-Loop Model Validation with Data

The open-loop behavior of the full in silico simulation model is compared to experimental data from the literature.

1) Baseline hemodynamics of nominal and hypertensive rats: The baseline parameters are identified to simulate healthy and hypertension conditions. The new steady state of the diseased model shows a significant increase in systolic arterial pressure and end diastolic pressure. A hypertensive rat also has a faster heart rate, preserved ejection fraction, smaller stroke volume, and smaller end diastolic volume than a healthy rat. The changes of the new steady state value in fold-change from healthy to diseased state are illustrated in FIG. 6, compared to data from [31]. The simulated P-V loops of the left heart for both healthy and disease states and the corresponding physiological outputs are shown in FIG. 7 and Table IV, respectively.

2) Hemodynamic response to exercise: In the simulation of a certain level of exercise, a step increase of sympathetic and vagal offset is introduced when exercise starts and the resistance of the active muscle progressively decreases following a first-order dynamic. The dynamic response of hemodynamic variables to exercise is illustrated in FIG. 8. The exercise starts around 9 s. The MAP increases and then decreases until it achieves a new steady state. The increase in MAP is caused by the overall effector response to nervous offset, while the decrease is due to the reduction in systemic arterial resistance (SVR). The new steady state consists of a higher arterial pressure, heart rate, stroke volume and cardiac output. These changes are shown in FIG. 9 (left) for a nominal rat and compared to experimental data from [32], [33].

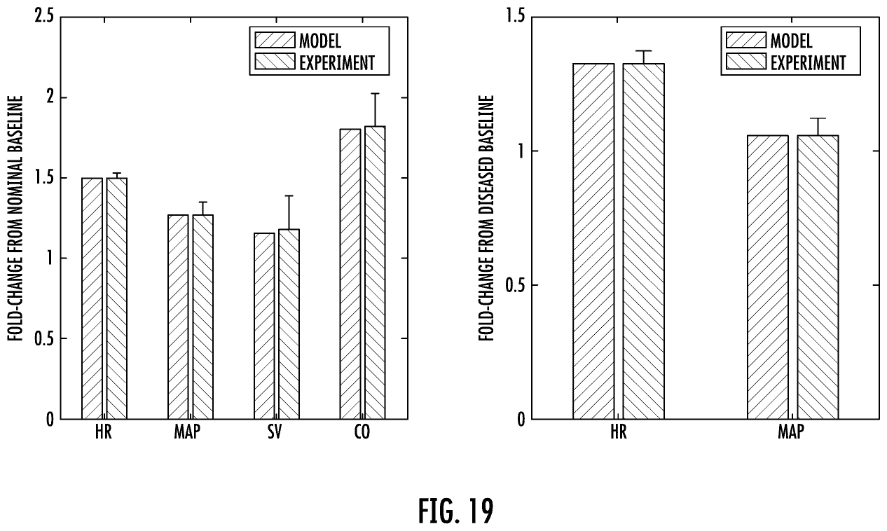

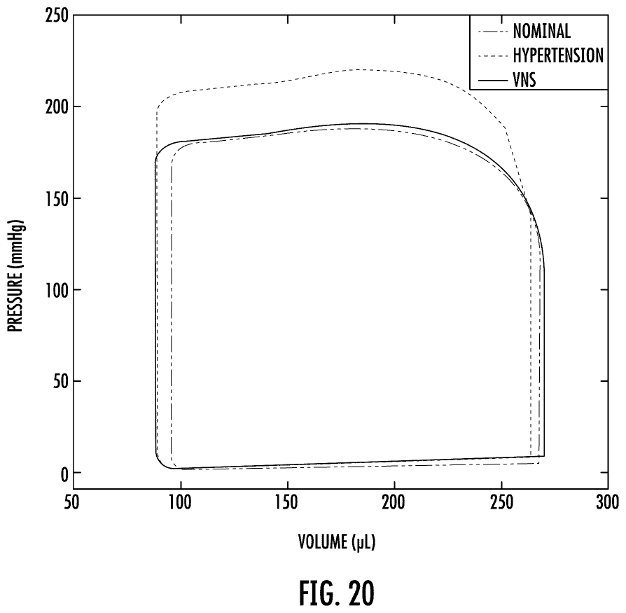

To simulate the exercise condition for a SHR, the parameters representing the constant gain factor that qualify the effect of sympathetic activity on total unstressed blood volume, heart period, left ventricular elastance are also modified. The dynamic response of the physiological outputs are similar to those of a nominal rat, which are not shown here. The changes of the steady state HR and MAP of a SHR is shown in FIG. 19 (right), compared to the experimental data from [33]. Although both HR and MAP increase compared with rest condition, the change is less significant compared to that of nominal rat. This is expected because sympathetic activity dominates in hypertensive rat [33] in rest condition and the increase in the sympathetic activity caused by exercise may bring the sympathetic activity to its upper saturation. The P-V loops and hemodynamic variables for nominal and hypertensive rats during exercise are shown in FIG. 20 and Table IV, respectively.

3) Effects of VNS on MAP and HR: To simulate the effect of VNS on MAP and HR in rats, the parameters representing the midpoint and slope of the activation curve, the weighing matrices of the conduction map, and the fiber distributions are identified to match experimental observations reported in [4], [30]. The parameters of the activation curve not only vary with fiber type, but also vary with stimulation location because the distance between the cuff electrode and the target fiber may vary between different stimulation locations.

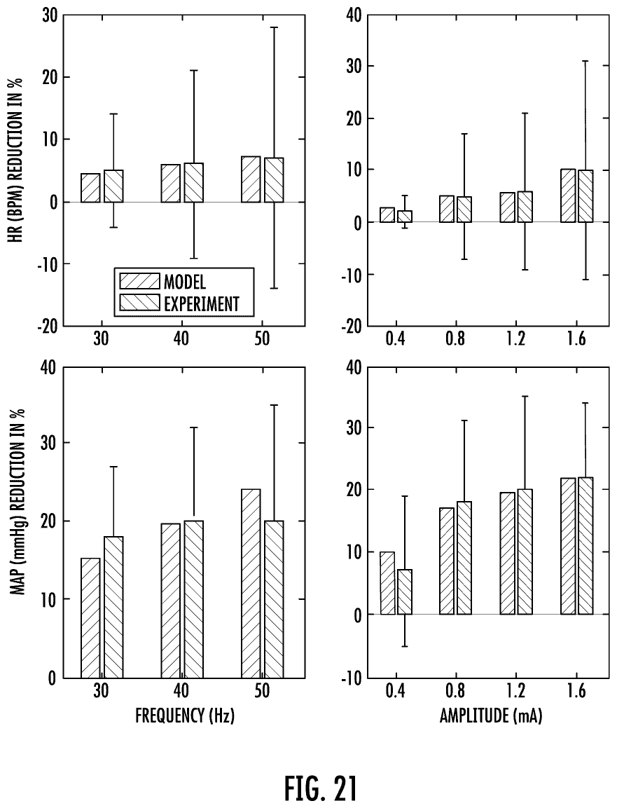

The response of the MAP and HR in percent as a function of stimulation frequency and stimulation amplitude for a barore-ceptive location are illustrated in FIG. 21, compared to that of experimental data from [30]. In this location, all stimulation parameters decreased the MAP and only the large amplitude causes obvious bradycardia. To reproduce this phenomenon, we set this location with 100% of barofibers and 10% of the total vagal fibers. Recruitment of the barofibers requires a small amplitude and primarily accounts for the decrease in MAP, while recruitment of the vagal fibers mainly accounts for the decrease in HR with large amplitude. The HR response matches well with the experimental data, while there is some mismatch for the effect of frequency on MAP. According to experiments, stimulations at 40 Hz had the strongest impact on the MAP, but the prediction from the model shows that the MAP continuously decreases with frequency, which suggests an upper saturation should be provided for fiber firing rate besides the linear conduction map.

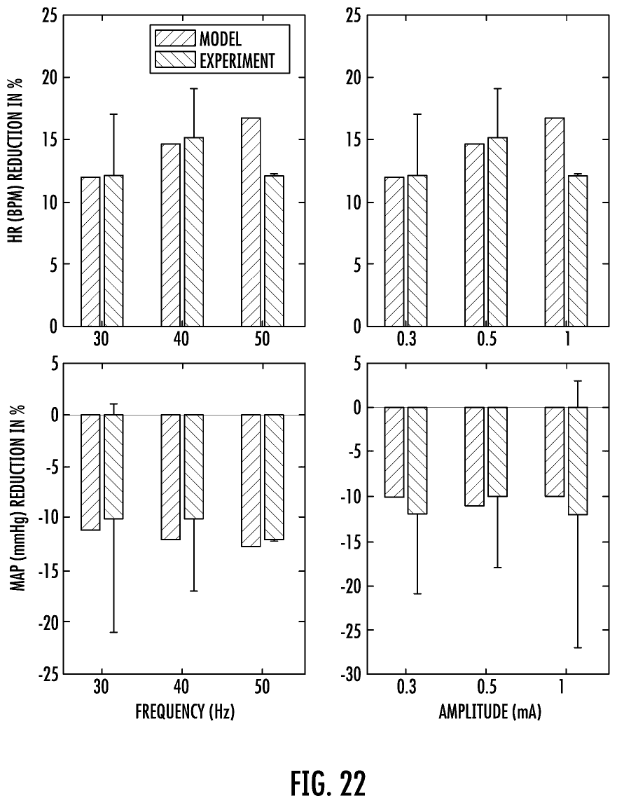

The response of the MAP and HR in percent for a non-baroreceptive location are illustrated in FIG. 22, compared to that of experimental data from [4]. Stimulation through a non-baroreceptive location introduces a small increase in MAP, re-gardless of parameter settings, but showed severe bradycardia. One reason for the increase in MAP is the recruitment of the sympathetic fibers, so 12% of total sympathetic fibers and 50% of total vagal fibers are set for the non-baroreceptive location. The third stimulation location are set to contain the other 40% of total vagal fibers. When considering the effect of multiple stimulation locations, we made a strong assumption that the

### B. Evaluation of NMPC

The NMPC algorithm is evaluated using three simulation scenarios: 1) tracking of nominal state, 2) disturbance rejection, 3) adaptation to exercise.

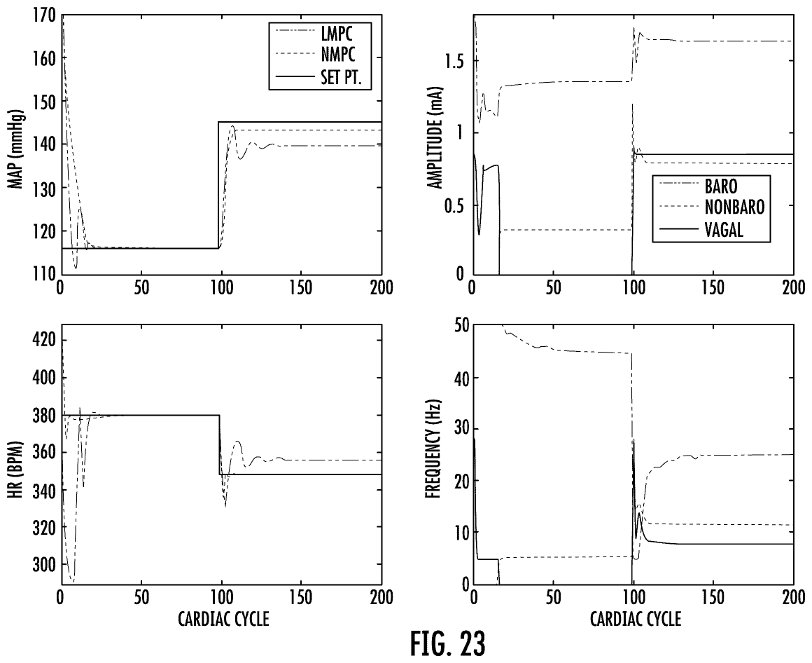

1) Evaluation of NMPC to track nominal state: Two types of set point tracking cases are used to evaluate the NMPC algorithm for both rest scenarios: (a) nominal set points, (b) non-nominal set points. FIG. 23 shows the input and output response of NMPC as well as the output response of MPC using a state space model linearized at the nominal points (LMPC in red) for each case. The ‘true’ system starts at its steady state simulating a hypertensive rat in rest state. In our set-point tracking study, no disturbance is estimated (Bd, Bpd, Cd matrices set to zero in (22)), and the steady state target calculator is only used at the first cycle when the regulator starts or when a setpoint change occurs. The simulation results showed that the NMPC gave better performance compared with LMPC. In case (a), both NMPC and LMPC converge with zero offset. However, overshoot is observed by using LMPC because a linear model lacks the ability to capture the dynamics far from the linearization point. In case (b), NMPC converges to the non-nominal setpoint with a much smaller offset compared with the LMPC.

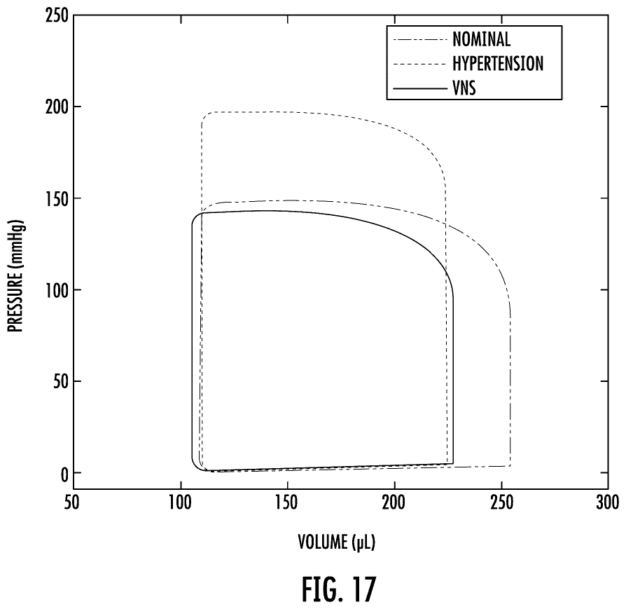

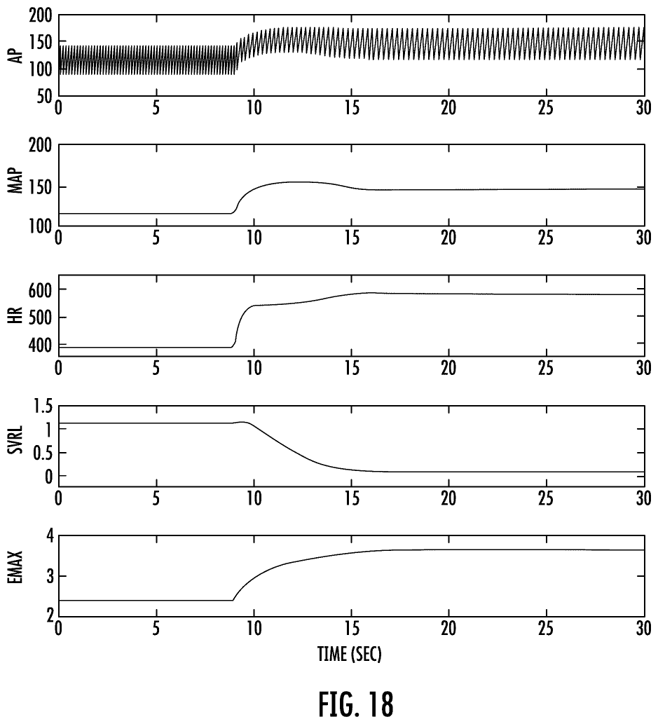

The convergence of a solution in the regulator problem is panelized by the terminal cost. Offset of the outputs are caused by the model mismatch, which can be reduced by reducing the weighting on unmeasured state and inputs in the regulator objective function. The modification of P-V loop and the corresponding hemodynamic outputs in rest state using optimal value of inputs calculated by NMPC are illustrated by the blue line in FIG. 17 and Table IV, respectively. It indicates that short-term VNS to regulate nominal HR and MAP may effectively release hypertension, but can not resolve diastolic heart failure condition.

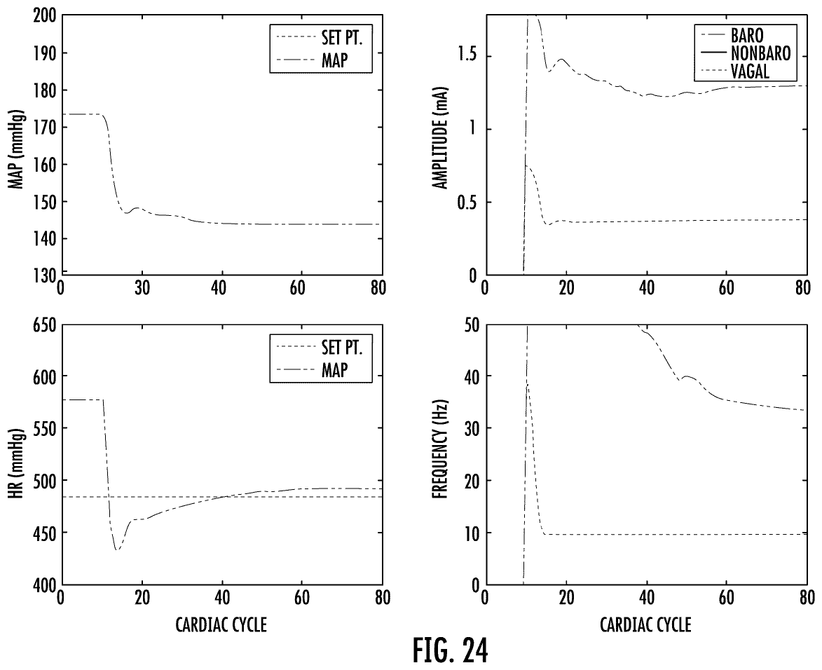

A nominal setpoint tracking case is applied for exercise regime. FIG. 24 shows the simulation results. None of the combinations of the stimulation locations are calculated as feasible by the target problem to track nominal value of HR and MAP in exercise state (in Table IV). This is expected because the activation of baroreceptive fibers to reduce blood pressure may also cause bradycardia, while the nominal value of HR is similar to the HR in diseased condition. So the setpoint of HR is set to 485 bpm, which allows a moderate reduction in HR, and the setpoint of the MAP is the same as its nominal value. The modification of P-V loop and the corresponding hemodynamic outputs in exercise state using optimal value of inputs calculated by NMPC are illustrated by the blue line in FIG. 20 and Table IV, respectively.

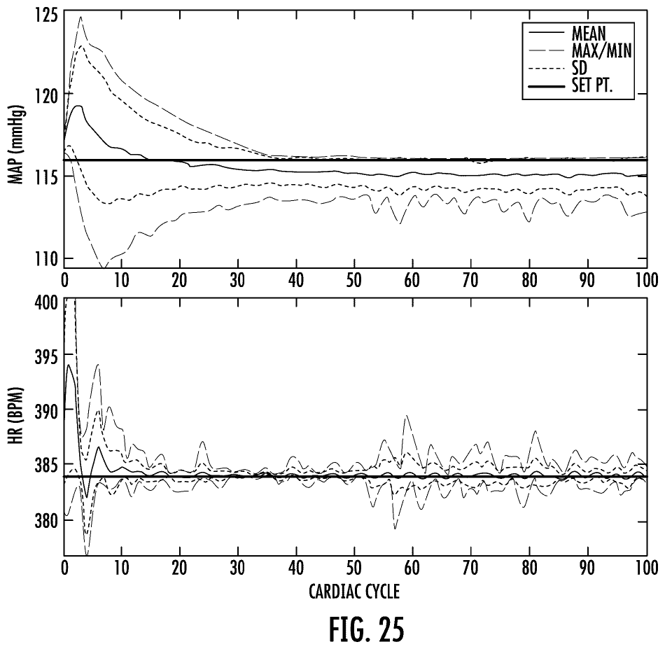

2) Evaluation of NMPC to disturbance rejection: One of the critical challenges for controlling a physiological system is the robustness against the inter and intra-subject variability in physiological parameters. In order to represent subject variability, two of the parameters involved in modeling the diseased state, representing the gain of the systemic resistance (GR) and the gain of the cardiac period (GT s) due to sympa-thetic drive, are changed. Specifically, we created a parameter space that spanned±5-fold range from nominal values of these two parameters to simulate 100 ‘true’ rats. Ten of the rats are randomly selected from the 100 examples to test the robustness of NMPC. The average and max/min output response using NMPC with disturbance estimation for ten rats in FIG. 25 indicates that our NMPC algorithm indeed works to move the HR and MAP to their desired setpoint even though there is a mismatch between the reduced model and the ‘true’ system.

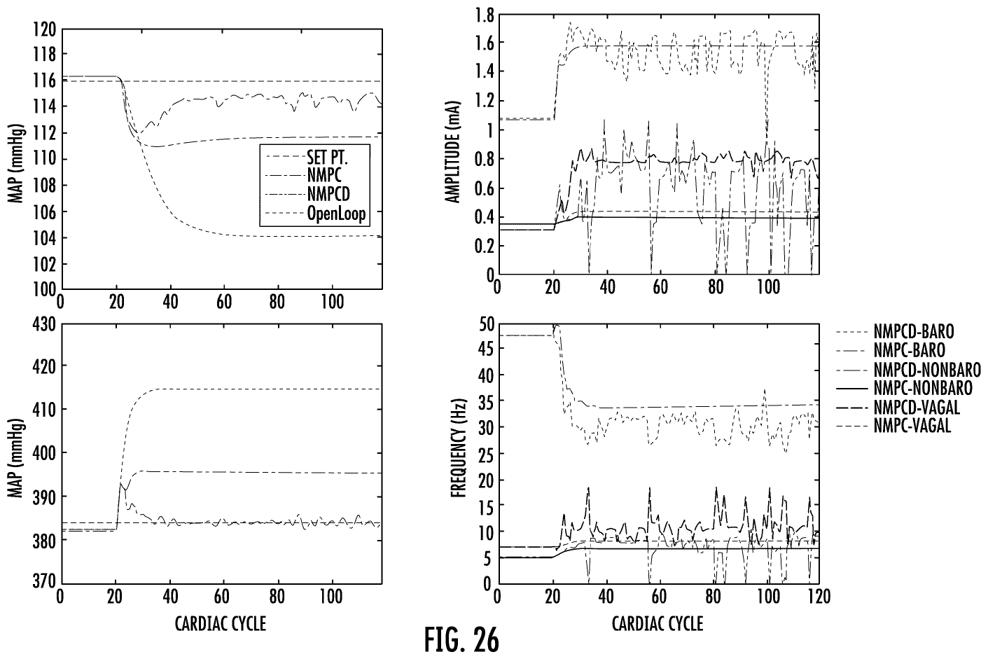

Also, one closed-loop example is shown in FIG. 26 to com-pare NMPC with disturbance estimation (solid line) with a standard NMPC algorithm (dashed line) as well as the open-loop performance (dotted line). In this case, disturbances are estimated for input and output variables, by setting nonzero values in the diagonal of Bd and Cd matrices. When the disturbance occurs at 20 cardiac cycles, both designs of the NMPC automatically adjust the input variables to move the output back to setpoints. The NMPC with disturbance estimation corrects for the disturbance with smaller offset than the other, but the input variables are with large oscillations and the system never reaches a steady state. In addition, a target calculation problem has to be repeated at each sample time due to the estimated disturbance, which makes the computation time much longer. A tradeoff between the number of disturbance variables, the computational expense, and the controller performance should be determined for real-time application.

3) Evaluation of NMPC for adaptation from rest to exercise scenario: During acute exercise, multiple physiological re-sponses are triggered that increase both the HR and MAP. Some control strategies should be considered by NMPC to modify stimulation signals before, during and after exercise. Here, two distinct ways are studied for adaptation to exer-cise state in NMPC design: 1) switch the controller with the exercise model and the corresponding setpoint, 2) treat the physiological activity as an acute disturbance, generating unpredicted changes in physiological parameters associated with shift in sympathetic and vagal balance, which can be estimated by the MHE.

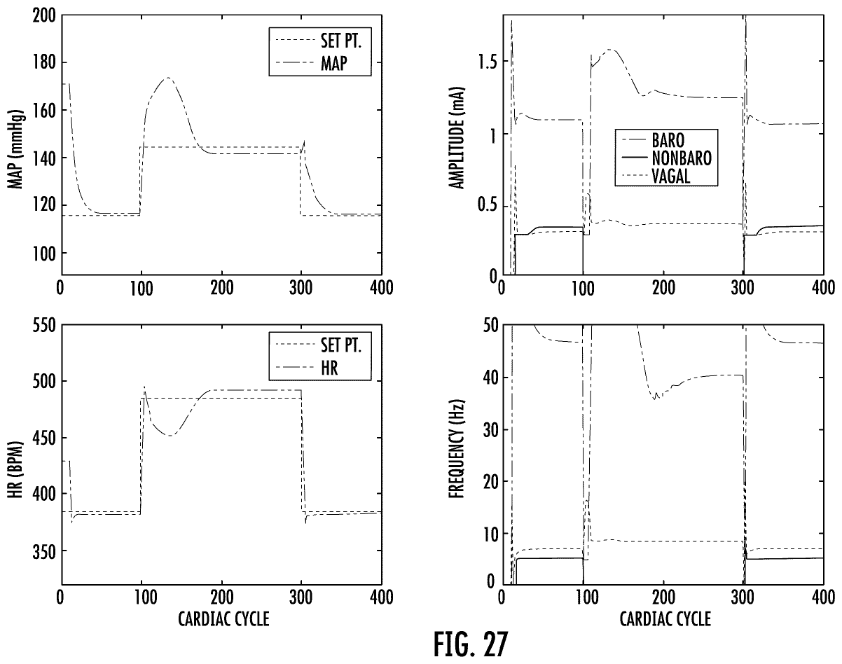

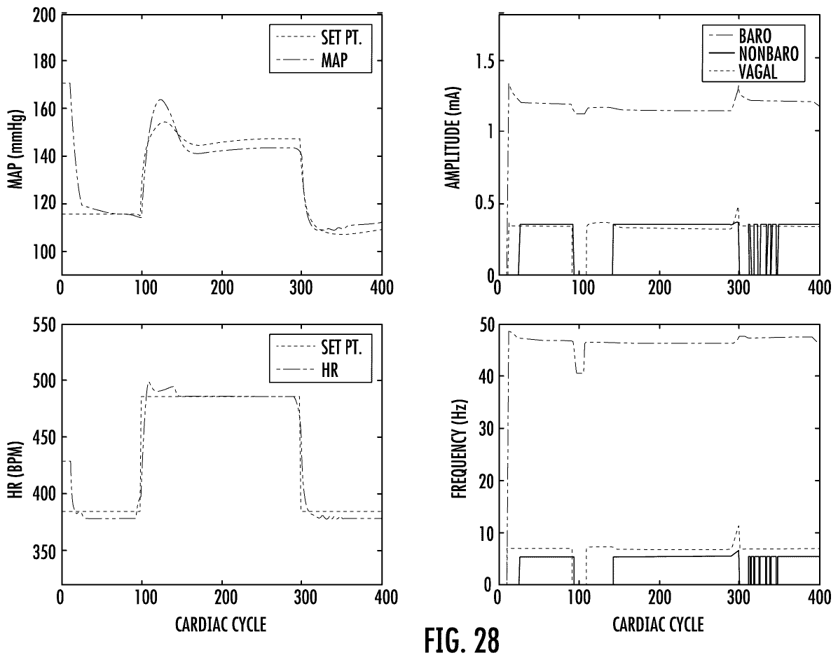

An example showing the performance of NMPC before, during and after exercise using the first method is illustrated in FIG. 27. When exercise starts at 100 cardiac cycle, the NMPC predicts the future dynamics of the system using the exercise model, then switches back to rest model at cardiac cycle 300. This kind of closed-loop design requires a large data set to identify an exercise model off-line. In addition, such a model can only capture the steady state dynamics, but can not predict the transition behavior between rest and exercise state. The other example showing the performance of MPC using the second strategy is illustrated in FIG. 28. A trajectory of nominal MAP from rest to exercise, then back to rest, instead of its steady state value are used in NMPC to capture its slow dynamics which is consistent with exercise physiology, while the same setpoint change is applied for HR because it has a much faster dynamics than MAP and the nominal HR during exercise cannot be achieved as discussed above. In this condition, the target problem cannot work and the objective function of the controller is modified to the standard formulation of MPC in (24). The MHE automatically estimates the change on the model parameters by setting nonzero values in Bpd matrices. The NMPC works well to track the trajectory without large modification of the stimulation configurations.

### DISCUSSION

A novel model-based multivariable control framework has been proposed to compute optimal parameters for closed-loop multi-location VNS. The advances resulting from this formulation are summarized below:
1) a general, experimentally validated model representing the CVS of a rat, with baroreflex regulation, was de-veloped to quantitatively characterize the effect of VNS parameters on hemodynamic response for various scenarios of normal and hypertension (disease) conditions as well as resting and exercise states;
2) multiple stimulation locations are explicitly incorporated in the controller design to exploit variable functional responses;
3) the variability in composition of the vagal nerve, particularly, the specific fascicles and the relative distribution of their functions—afferent versus efferent, sympathetic versus parasympathetic—is explicitly captured quantitatively in the model by assigning varying parameter values for the fiber concentration in different stimulation locations, slope of activation curve and conduction reliability function.
4) the proposed model quantitatively captures the effect of stimulation intensity as a percent recruitment of nerve fibers, while the effect of the stimulation frequency is captured using a conduction map to account for the interaction between internal and external electrical signals.
5) the proposed novel controller uses a cycle-averaged model to control both HR and MAP by adjusting several stimulation parameters in different stimulation locations simultaneously in a truly multivariable context, account-ing for coupling and nonlinearities.
6) the performance of the controller is tested for its ability to reject disturbances and handle inter and intra-subject variability, in realistic scenarios describing conditions prior to hypertension.
   The proposed closed-loop design can be generalized to other MIMO control objectives. While our current study is directed to regulate nominal HR and MAP for hypertension related cardiac disease, VNS has been demonstrated as therapeutic for a multitude of diseases due to its long-term beneficial effects. In these cases, long-term mechanisms need to be captured by the model. Additionally, other biomarkers, such as heart rate variability and baroreflex sensitivity, may be more important than HR and MAP in the control objective. Future research activities are focused on optimization of the proposed control framework with an online adaptation approach and integration in an embedded hardware for real-time demonstration.

### REFERENCES

[1] Arimura, Takahiro, et al., “Intravenous electrical vagal nerve stimulation prior to coronary reperfusion in a canine ischemia-reperfusion model markedly reduces infarct size and prevents subsequent heart failure,” International Journal of Cardiology, vol. 227, pp. 704-710, 2017.
[2] Li, Meihua, et al., “Vagal nerve stimulation markedly improves long-term survival after chronic heart failure in rats,” Circulation, vol. 109, no. 1, pp. 120-124, 2004.
[3] Carpenter, Alexander, et al., “Vagal atrial fibrillation: what is it and should we treat it?” International journal of cardiology vol. 201, pp. 415-421, 2015.
[4] Plachta, Dennis T T, et al., “Blood pressure control with selective vagal nerve stimulation and minimal side effects,” Journal of neural engineering, vol. 11, no. 3, p. 036011, 2014.
[5] Premchand, Rajendra K., et al., “Autonomic regulation therapy via left or right cervical vagus nerve stimulation in patients with chronic heart failure: Results of the ANTHEM-HF trial,” Journal of Cardiac Failure, vol. 20, no. 11, pp. 808-816, 2014.
[6] Zannad, Faiez, et al., “Chronic vagal stimulation for the treatment of low ejection fraction heart failure: Results of the neural cardiac therapy for heart failure (NECTAR-HF) randomized controlled trial,” European Heart Journal, vol. 36, no. 7, pp. 425-433, 2015.
[7] Gold, Michael R., et al., “Vagus nerve stimulation for the treatment of heart failure: The INOVATE-HF trial,” Journal of the American College of Cardiology, vol. 68, no. 2, pp. 149-158, 2016.
[8] Waninger, Matthew S., et al., “Electrophysiological control of ventricular rate during atrial fibrillation,” Pacing and Clinical Electrophysiology, vol. 23, no. 8, pp. 1239-1244, 2000.
[9] Zhang, Youhua, et al., “Optimal ventricular rate slowing during atrial fibrillation by feedback av nodal-selective vagal stimulation,” American Journal of Physiology-Heart and Circulatory Physiology, vol. 282, no. 3, pp. H1102-H1110, 2002.
[10] Tosato, Marco, et al., “Closedloop control of the heart rate by electrical stimulation of the vagus nerve,” Medical and Biological Engineering and Computing, vol. 44, no. 3, pp. 161-169. 2006.
[11] Ugalde, Hector M. Romero, et al., “Model-based design and experimen-tal validation of control modules for neuromodulation devices,” IEEE Transactions on Biomedical Engineering, vol. 63, no. 7, pp. 1551-1558, 2015.
[12] Romero-Ugalde, Hector M., et al., “A novel controller based on state-transition models for closed-loop vagus nerve stimulation: Application to heart rate regulation,” PloS one, vol. 12, no. 10, p.eo186068, 2017.
[13] Ojeda, David, et al., “Sensitivity analysis of vagus nerve stimulation parameters on acute cardiac autonomic responses: Chronotropic, inotropic and dromotropic effects,” PloS one, vol. 11, no. 9 p. eo163734, 2016.
[14] Rao, Ramesh R., Brian Aufderheide, and B. Wayne Bequette, “Ex-perimental studies on multiple-model predictive control for automated regulation of hemodynamic variables,” IEEE Transactions on biomedical engineering, vol. 50, no. 3, pp. 277-288, 2003.
[15] Dua, Pinky, Francis J. Doyle, and Efstratios N. Pistikopoulos, “Model-based blood glucose control for type 1 diabetes via parametric program-ming,” IEEE Transactions on Biomedical Engineering, vol. 53, no. 8, pp. 1478-1491, 2006.
[16] Kirsch, Nicholas, Naji Alibeji, and Nitin Sharma, “Nonlinear model pre-dictive control of functional electrical stimulation,” Control Engineering Practice, vol. 58, pp. 319-331, 2017.
[17] Yao, Yuyu, and Mayuresh V. Kothare, “Model Predictive Control of Se-lective Vagal Nerve Stimulation for Regulating Cardiovascular System,” 2020 American Control Conference, pp. 563-568, 2020.
[18] Williams, Nakeya D., et al., “Cardiovascular dynamics during head-up tilt assessed via pulsatile and non-pulsatile models,” Journal of mathematical biology, pp. 987-1014, 2019.
[19] Ursino, Mauro, “Interaction between carotid baroregulation and the pul-sating heart: a mathematical model,” American Journal of Physiology-Heart and Circulatory Physiology, vol. 275, no. 5, p. H1733-H1747, 1998.
[20] Mahdi, Adam, et al., “Modeling the afferent dynamics of the barore-flex control system,” PLoS computational biology, vol. 9, no. 12, p. e1003384, 2013.
[21] Sturdy, Jacob, Johnny T. Ottesen, and Mette S. Olufsen, “Modeling the differentiation of A- and C-type baroreceptor firing patterns,” Journal of Computational Neuroscience, vol. 42, no. 1, pp. 11-30, 2017.
[22] Seki, Atsuko, et al., “Sympathetic nerve fibers in human cervical and thoracic vagus nerves,” Heart Rhythm, vol. 11, no. 8, pp. 1411-1417, 2014.
[23] Kawada, Toru, et al., “Contribution of afferent pathway to vagal nerve stimulation-induced myocardial interstitial acetylcholine release in rats,” American Journal of Physiology-Regulatory, Integrative and Compar-ative Physiology, vol. 319, no. 5, pp. R517-R525, 2020.
[24] Settell, Megan L., et al., “Functional vagotopy in the cervical vagus nerve of the domestic pig: implications for the study of vagus nerve stimulation,” Journal of neural engineering, vol. 17, no. 2, p. 026022, 2020.
[25] Stauss, H. M., “Stauss, Harald M.” Differential hemodynamic and respiratory responses to right and left cervical vagal nerve stimulation in rats,” Physiological reports, vol. 5, no. 7, p. e13244, 2017.
[26] Ardell, J. L., and Randall, W. C., “Selective vagal innervation of sinoatrial and atrioventricular nodes in canine heart,” American Journal of Physiology-Heart and Circulatory Physiology, vol. 251, no. 4, pp. H764-H773, 1986.
[27] Bucksot, Jesse E., et al., “Flat electrode contacts for vagus nerve stimulation,” PloS one, vol. 14, no. 11, p. eO215191, 2019.
[28] Sadashivaiah, Vijay, et al., “Modeling the interactions between stimu-lation and physiologically induced APs in a mammalian nerve fiber: dependence on frequency and fiber diameter,” Journal of computational neuroscience, vol. 45, no. 3, pp. 193-206, 2018.
[29] Pacher, Pa'l, et al., “Left ventricular pressure-volume relationship in a rat model of advanced aging-associated heart failure,” American Journal of Physiology-Heart and Circulatory Physiology, 2004.
[30] Gierthmuehlen, Mortimer, and Dennis T T Plachta, “Effect of selective vagal nerve stimulation on blood pressure, heart rate and respiratory rate in rats under metoprolol medication,” Hypertension Research, vol. 39, no. 2, pp. 79-87, 2016.
[31] Cingolani, Oscar H., et al., “Increased systolic performance with dias-tolic dysfunction in adult spontaneously hypertensive rats.” Hyperten-sion, vol. 41, no. 2, pp. 249-254, 2003.
[32] Flaim, STEPHEN F., et al., “Cardiovascular response to acute aquatic and treadmill exercise in the untrained rat.” Journal of Applied Physi-ology, vol. 46, no. 2, pp. 302-308, 1979.
[33] Chandler, Margaret P., and Stephen E. Dicarlo. “Acute exercise and gender alter cardiac autonomic tonus differently in hypertensive and nor-motensive rats.” American Journal of Physiology-Regulatory, Integrative and Comparative, vol. 274, no. 2, pp. R510-R516, 1998.
[34] Lau, Kevin D., and C. Alberto Figueroa, “Simulation of short-term pres-sure regulation during the tilt test in a coupled 3D-oD closed-loop model of the circulation.” Biomechanics and modeling in mechanobiology, vol. 14, no. 4, pp. 915-929, 2015.
[35] Ottesen, J. T., Olufsen, M. S., and Larsen, J. K., “Applied mathematical models in human physiology.” Society for Industry and Applied Math-ematics, 2004.

1-53. (canceled)

54. A computer program product encoded on a computer-readable storage medium comprising instructions for:
(a) measuring the mean arterial pressure (MAP) in a given cardiac cycle;
(b) measuring the heart rate (HR) in a given cardiac cycle;
(c) predicting a control cardiac response of the circulatory loop with at least a first control criterion over a control time period;
(d) calculating a desired adjustment value of the stimulation parameters for MAP and/or HR to approach the control cardiac response;
(e) executing a signal command to stimulate the vagal nerve with an electrical pulse sufficient to adjust the MAP and/or HR in real-time with a magnitude corresponding to the desired adjustment value at a first, second and third location within a circulatory loop.

55. The computer program product according to claim 54, wherein step (c) comprises: (i) applying a piece-wise linear or multiple local linear functions corresponding to interaction of MAP and HR within the circulatory loop.

56. The computer program product according to claim 54, wherein step (c) comprises: (ii) determining the probability of accomplishing the control cardiac response using a switch function.

57. The computer program product according to claim 54, wherein step (d) comprises: (iii) calculating the weight of the step of predicting using the measured values of (a) and (b).

58. The computer program product according to claim 54, wherein step (d) comprises: (iv) calculating the total frequency of action potentials sufficient to adjust the MAP and/or HR in real-time with a magnitude corresponding to the desired adjustment value, wherein the total frequency of action potentials is based upon a modeled output value of step (c) and the real-time measured values of steps (a) and (b).

59. The computer program product according to claim 54, further comprising: step (f) repeating steps (a) through (e) over a set time period for continuous monitoring of HR and MAP.

60. The computer program product according to claim 54, wherein step (e) comprises: adjusting pulse amplitude and pulse frequency across the first, second and third locations of the circulatory loop to alter HR and MAP.

61. The computer program product according to of claim 60, wherein at least one of the first, second or third locations is a nerve fiber on the vagal nerve.

62. The computer program product according to claim 55, wherein the piece-wise linear function comprises
$${x}^{i} ( k + 1 ) = {A}^{i} {x}^{i} ( k ) + {B}^{i} u ( k ) + {Bd}^{i} {d}^{i} ( k ) .$$
$${y}^{i} ( k ) = {C}^{i} {x}^{i} ( k ) + {D}^{i} u ( k ) + {d}^{i} ( k ) ,$$
wherein the superscript i represents the model number; di(k) is assumed Gaussian noise with zero mean imposed on the outputs, Aⁱ, Bⁱ, Cⁱ, Dⁱ are operating ranges of MAP in a cardiac cycle, k is the cardiac cycle number in which the numbers are being calculated, x is the operating region in cycle k, and y is the operating region in cycle k+1, u is an input value of MAP.

63. The computer program product according to claim 57, wherein the desired adjustment value of the stimulation parameters is calculated by formula:
$$\begin{matrix}\begin{matrix}min \\ u ( k + i \vert k )\end{matrix} \\ i = 1 , 2 , … , {N}_{c}\end{matrix} \qquad {\sum }_{i = 0}^{N} \text{?} ( {‖ \hat{y} ( k + i \vert k ) − r ( k + i \vert k ) ‖}_{Q}^{2} + {\sum }_{i = 1}^{N} \text{?} {‖ u ( k + i \vert k ) - u ( k + i - \text{?} \vert k ) ‖}_{R}^{2} + {\sum }_{i = 1}^{N} \text{?} {‖ u ( k + i \vert k ) - u \text{?} ( k + i \vert k ) ‖}_{P}^{2} \text{?}$$
$$\text{?} \text{indicates text missing or illegible when filed}$$
wherein Nc is the number of cardiac cycles in a control horizon; wherein k+ilk is prediction into future cardiac cycle number time k+i based
on the measurement at current sampling instance k; yA is the estimated output number, r is the set point, u_{b} is the baseline input of MAP; and wherein Q is the output weight matrix; R is the input weight matrix; and P is the integral action.

64. A system comprising:
(i) the computer program product according to claim 54; and
(ii) a processor operable to execute programs; and/or
a memory associated with the processor.

65. A system for identifying modulating HR and/or MAP comprising:
(i) a processor operable to execute programs;
(ii) a memory associated with the processor;
(iii) a database associated with and operably connected to said processor and said memory;
(iv) a computer program product stored in the memory and executable by the processor, the program being operable for:
(a) measuring a mean arterial pressure (MAP) in a given cardiac cycle within a circulatory loop of the subject;
(b) measuring a heart rate (HR) in a given cardiac cycle within a circulatory loop;
(c) predicting a control cardiac response of the circulatory loop with at least a first control criterion over a control time period;
(d) calculating a desired adjustment value of the stimulus parameters for MAP and/or HR to approach the control cardiac response;
(e) executing a signal command to stimulate the vagal nerve with an electrical pulse sufficient to adjust the MAP and/or HR in real-time with a magnitude corresponding to the desired adjustment value at a first, second and third location within the vagal nerve; and
(v) an implantable device comprising the computer program product and at least a first, second, and third electrode in operable electrical communication with the processor.

66. The system according to claim 65, wherein the computer program product is operable for step (c) by applying a piece-wise linear or multiple local linear functions corresponding to interaction of MAP and HR within the circulatory loop.

67. The system according to claim 65, wherein the computer program product is operable for step (c) by (ii) determining the probability of accomplishing the control cardiac response using a switch function.

68. The system according to claim 65, wherein the computer program product is operable for step (d) by (iii) calculating the weight of the step of predicting using the measured values of (a) and (b).

69. The system according to claim 65, wherein the computer program product is operable for step (d) by (iv) calculating the total frequency of action potentials sufficient to adjust the MAP and/or HR in real-time with a magnitude corresponding to the desired adjustment value, wherein the total frequency of action potentials is based upon a modeled output value of step (c) and the real-time measured values of steps (a) and (b).

70. The system according to claim 65, wherein the computer program product is further operable for: (f) repeating steps (a) through (e) over a set time period for continuous monitoring of HR and MAP.

71. The system according to claim 65, wherein the computer program product is operable for step (e) by adjusting pulse amplitude and pulse frequency across the first, second and third locations of the circulatory loop.

72. The system according to claim 65, wherein the device comprises the controller and the computer program product.

73. The system according to claim 65, wherein the desired adjustment value for the stimulus parameters is calculated by the computer program product by formula:
$$\begin{matrix}\begin{matrix}min \\ u ( k + i \vert k )\end{matrix} \\ i = 1 , 2 , … , {N}_{c}\end{matrix} \qquad {\sum }_{i = 0}^{N} \text{?} ( {‖ \hat{y} ( k + i \vert k ) − r ( k + i \vert k ) ‖}_{Q}^{2} + {\sum }_{i = 1}^{N} \text{?} {‖ u ( k + i \vert k ) - u ( k + i - \text{?} \vert k ) ‖}_{R}^{2} + {\sum }_{i = 1}^{N} \text{?} {‖ u ( k + i \vert k ) - u \text{?} ( k + i \vert k ) ‖}_{P}^{2} \text{?}$$
$$\text{?} \text{indicates text missing or illegible when filed}$$
wherein Nc is the number of cardiac cycles in a control horizon; wherein k+ilk is prediction into future cardiac cycle number time k+i based on the measurement at current sampling instance k; yA is the estimated output number, r is the set point, u_{b} is the baseline input of MAP; and wherein Q is the output weight matrix; R is the input weight matrix; and P is the integral action.
